(19) United States
(12) Patent Application Publication
(10) Pub. No.: US 2002/0143164 A1
(43) Pub. Date: Oct. 3, 2002

(54) Ras activator nucleic acid molecules, polypeptides and methods of use

(76) Inventors: Daniela Rotin, Thornhill (CA); Nam Pham, Thornhill (CA)

Correspondence Address: CHERYL H AGRIS PHD, PO BOX 806, PELHAM, NY, 10803, US

(21) Appl. No.: 09/911,826

(22) Filed: Jul. 20, 2001

Related U.S. Application Data
(63) Continuation of application No. PCT/CA00/00042, filed on Jan. 20, 2000.

(30) Foreign Application Priority Data
Jan. 20, 1999 (CA) 2,259,830

(51) Int. Cl. C07H 21/02; C07H 21/04

(52) U.S. Cl. 536/23.1

(57) ABSTRACT

The invention is a GRF4 nucleic acid molecule and its corresponding protein which has an important role in cell signaling. The invention also includes biologically functional equivalent nucleic acid molecules and proteins. The invention also relates to methods of using these nucleic acid sequences and proteins in medical treatments and drug screening.

### FIELD OF THE INVENTION

The invention relates to isolated nucleic acid molecules encoding Ras activator polypeptides. The invention also includes methods of using the polypeptides and nucleic acid molecules and proteins for treatment of cancer and neuronal diseases, disorders and abnormal physical states.

### BACKGROUND OF THE INVENTION

Activation of the Ras signaling pathway controls numerous cellular functions, most notably those regulating cell proliferation, differentiation and transformation. Ras is involved in many aspects of cellular metabolism, so modulation of Ras activity and concentration provides a mechanism to control many cellular disease, disorders and abnormal physical states, such as cancer.

To date, 3 classes of Guanine Nucleotide Exchange/Releasing Factors (GEFs/GRFs) which activate Ras have been identified: (i) SOS, which binds Grb2 and connects growth factor receptors to Ras, (ii) Ras GRF½, which contains an IQ motif and is activated in response Ca2+/calmodulin, and (iii) RasGRP, which contains a diacylglycerol binding domain and an EF hand, and is activated by diacylglycerol and Ca2+.

None of the known classes of Ras activators have been satisfactorily modulated to control human cellular pathology. There is a clear need to identify new ways to control Ras concentration and activity.

### SUMMARY OF THE INVENTION

The invention relates to a Ras activator, GRF4. This activator contains several domains, including CDC25, REM, RA, PDZ and a cNMP (cAMP/cGMP) binding domain (cNMP-BD), 2 PY motifs and a C terminal SxV sequence. GRF4 can activate Ras in vitro or in vivo. It binds CAMP directly via its cNMP-BD. GRF4 directly connects CAMP-generating (e.g. G protein coupled receptors) or cGMP-generating pathways to Ras. GRF4 is expressed mainly in the brain, and is localized at the plasma membrane, a localization dependent on the presence of intact PDZ domain.

Using an expression library screen of mouse embryonic library with the second WW domain of Nedd4 as a bait, we identified Clone 7.7, encoding about 150 amino acids, which bear 75% identity and 95% similarity to KIAA0313, a human Clone (encoding an approximately 1500 amino acid protein) deposited in Genbank. The segment we isolated contained 2 PY motifs (xPPxY) which are responsible for the binding to the Nedd4-WW domain. We identified the following domains (by sequence alignment) in clone KIAA0313, and hence renamed it GRF4, because it represents the fourth class of Ras activators: a CDC25 homology domain (most similar to yeast CDC25 and SDC25, Ras GRF½ and SOS), a PDZ domain, a cNMP binding domain (preferably cAMP-BD or cGMP-BD), a REM (Ras exchange motif) domain, a RA (Ras associating) domain, 2 PY motifs and a C terminal SAV sequence conforming to PDZ binding motif (SxV*, where * denotes STOP codon). The CDC25 of GRF4 domain has an approximately 40 amino acid insert, which includes a PKA phosphorylation site.

GRF4 schematic domain organization:

--cNMP-BD---REM---PDZ---RA---CDC25---PY-PY---SxV

The invention includes nucleic acid molecules and polypeptides (capable of activating ras) having this domain organization.

We have so far demonstrated:

(i) GRF4 binds cAMP (and cGMP) directly.

(ii) GRF4 activates Ras in vitro and in living cells. In cells, GRF4 activates Ras in response to elevation of intracellular cAMP or cGMP.

(iii) GRF4 forms a stable complex with Ras in vitro.

(iv) GRF4 mRNA is expressed mainly in the brain (most brain regions) and GRF4 protein is expressed in brain lysates and synaptosomes.

(v) The function of the cNMP-BD of GRF4 is to entrance activation of GRF4 following cAMP or cGMP binding. Treatment of HEK-293T cells transfected with GRF4 with membrane permeant analogues of cAMP (8-bromo-cAMP) and cGMP (8-bromo-cGMP), or with agents that lead to elevation of intracellular levels of cAMP (Forskolin and IBnx) or cAMP (YC-1 and dipyridamole) leads to activation of Ras in GRF4-expressing cells but not in untransfected cells, demonstrating that these cNMP analogues can activate Ras via GRF4. Moreover, a mutant GRF4 in which the cNMP-binding domain (cNMP-BD) or the CDC25 domain is deleted fails to activate Ras.

(vi) GRF4 dimerizes or folds over itself. The PDZ domain of GRF4 can bind its own SAV sequence.

(vii) GRF4 is localized to the plasma membrane (where Ras is located), but is mislocalized in PDZ-mutated GRF4. The PDZ domain is responsible for targeting/localization of GRF4 at the plasma membrane. Inhibition of GRF4 or Ras can reduce cellulose proliferation and cancer.

(viii) GRF4 is a target for Nedd4 ubiquitination, as it binds Nedd4.

Due the presence of both cNMP-BD and a PDZ domain in GRF4, GRF4 connects G protein coupled receptors to Ras and thus to downstream signaling effectors of Ras, such as Raf-MAPK pathway, PI-3 kinase, raIGEF and possibly other effectors. G protein coupled receptors, a number of which contain a C terminal PDZ binding motif, activate adenylate cyclase via heterotrimeric G proteins, leading to increased cAMP. Thus, GRF4 binds via its PDZ to these receptors at the plasma membrane and the released cAMP directly activates GRF4 and thus stimulate Ras activation. When cGMP is the compound binding and activating GRF4, RasGRF may directly connect upstream activators of cGMP release (e.g. nitric oxide) to Ras. Nedd4 may regulate the stability of this protein by ubiquitination, and thus suppress GRF4 activity by regulating its stability and degradation.

The invention includes an isolated nucleic acid molecule encoding a polypeptide having GRF4 activity, preferably including all or part of the nucleic acid molecule of [SEQ ID NO: 1]. In another embodiment, the invention includes an isolated nucleic molecule having at least 40% sequence identity to all or part of the nucleic acid molecule of [SEQ ID NO: 1], wherein the nucleic acid molecule encodes a polypeptide having GRF4 activity.

Another embodiment is a nucleic acid molecule encoding all or part of the amino acid sequence of [SEQ ID NO: 2]. The invention also includes a nucleic acid molecule that encodes all or part of a GRF4 polypeptide or a polypeptide having GRF4 activity, wherein the sequence hybridizes to the nucleic acid molecule of all or part of [SEQ ID NO: 1] under high stringency conditions.

The invention includes an isolated polypeptide having GRF4 activity and a CDC25 domain, preferably, comprising all or part of the sequence of [SEQ ID NO: 2]. The polypeptide preferably comprising at least 40% sequence identity to all or part of the polypeptide of [SEQ ID NO: 2], wherein the polypeptide has GRF4 activity.

The invention includes a mimetic of the isolated polypeptide of any of claims 8 to 10, wherein the mimetic has GRF4 activity. Another aspect relates to a recombinant nucleic acid molecule comprising a nucleic acid molecule of the invention and a promoter region, operatively linked so that the promoter enhances transcription of the nucleic acid molecule in a host cell. The invention also includes a system for the expression of GRF4, comprising an expression vector and a nucleic acid molecule of the invention molecule inserted in the expression vector. The invention also includes a cell transformed by the expression vector of the invention. Another aspect of the invention relates to a method for expressing polypeptide by transforming an expression host with an expression vector including and culturing the expression host.

The invention also includes a pharmaceutical composition, including all or part of the polypeptide or mimetic of the invention, and a pharmaceutically acceptable carrier, auxiliary or excipient. Another aspect of the invention relates to a GRF4 specific antibody targeted to a region selected from the group consisting of the C-terminus, the CDC25 domain and the PDZ domain.

The invention includes a method of medical treatment of a disease, disorder or abnormal physical state, characterized by excessive GRF4 expression, concentration or activity, comprising administering a product that reduces or inhibits GRF4 polypeptide expression, concentration or activity. The invention also includes a method of medical treatment of a disease, disorder or abnormal physical state, characterized by inadequate GRF4 expression, concentration or activity, comprising administering a product that increases GRF4 polypeptide expression, concentration or activity.

### BRIEF DESCRIPTION OF THE DRAWINGS

Preferred embodiments of the invention will be described in relation to the drawings in which:

FIG. 1. Domain organization of Rat Nedd4.

FIG. 2. Protein sequence of Clone 7.7, the homolog of human clone KIAA0313.

FIG. 3A. Schematic Diagram of GRF4.

FIG. 3B. Shows the nucleic acid molecule that is [SEQ ID NO: 1] and the polypeptide that is [SEQ ID NO:2]. In a preferred embodiment, the figure shows GRF4.

FIG. 4A. Protein sequence alignment of CDC25 domains from several RasGEF/GRF including GRF4. The CDC25 domain of human GRF4 (hGRF4) was aligned with those of Drosophila GRF4 (dGRF4, identified from genomic DNA sequence [Accession number. AC005285, nucleotide sequence 122129-174319]), human Epac (hEpac), mouse RasGRF2 (mRasGRF2), Drosophila SOS (dSOS) and RasGRP (hRasGRP). The three structurally conserved regions present in CDC25 domains are lighter. Both hGRF4 and dGRF4 contain a unique insertion shown in blue. Alignments were created using the program Clustal W(1.7).

Accession numbers.

hGRF4 (AB002311), dGRF4(AC005285), hEpac(AF103905), mRasGRF2(U67326), dSOS(M83931), hRasGRP(AF106071), rLin-7-C(AF090136), hPTP-BAS-1(D21209), hDig(U61843), hPRKAR1B(M65066), hPSD-95 (AF1 56495), hPKGII(CAA76073), mEAG(U04294).

FIG. 4B. Comparison of CDC25 domain of GRF4 with RasGRF2 revealing the insert in GRF4.

FIG. 5. Protein sequence of alignment of Ras GRF4-REM domain.

FIG. 6A. Overall structure comparison between GRF4 and other known mammalian GRFs/GEFs which activate Ras.

FIG. 6B. An example of the most well known Ras signaling pathway.

FIG. 7. Sequence alignment of GRF4-PDZ domain. The PDZ domains of hGRF4 and dGRF4 were aligned with those of rat Lin-7-C (rLin-7-C), human PTP-BAS type 1 (hPTP-BAS-1), human Dig (hDig) and human PSD-95 (hPSD-95). The sequences corresponding the GLGF motif present in prototypic PDZ domains are lighter. GRF4 Alignments were created using the program Clustal W(1.7).

FIG. 8. Sequence alignment of GRF4-cNMP-BD. The cNMP-BD of hGRF4 was aligned with those of dGRF4, hEpac, human cAMP-dependent protein kinase regulatory subunit type 1b (hPRKAR1 B), human cGMP dependent protein kinase (hPKGII), and mouse cyclic nucleotide gated potassium channel (mEAG). The conserved motifs RAA present in hPRKAR1B and hEpac that confers cAMP binding specificity are shaded in blue. The conserved motifs RTA present in hPKGII and mEAG that confers cGMP binding specificity are lighter. Alignments were created using the program Clustal W(1.7).

FIG. 9. Protein sequence alignment of GRF4-RA domain.

FIG. 10. Tissue Distribution of GRF4.

FIG. 11. Co-precipitation of endogenous Nedd4 in Hek 293T cells by a GST-fusion 30 protein of the C-terminal last 150 aa of GRF4 which contains the two PY motifs.

FIG. 12. Co-immunoprecipitation of GRF4 with endogenous Nedd4 in Hek 293T cells transiently transfected with Flag-tagged GRF4.

FIG. 13. Method used for the in vitro GEF assay.

FIG. 14. In vitro GEF assay using immunoprecipitated full-length GRF4 demonstrating activation of Ras by GRF4 (additional data in FIG. 23(e)).

FIG. 15. GRF4 forms stable complex with GST-Ras in vitro.

FIG. 16. GRF4 induces foci formation in Rat2 fibroblasts.

FIG. 17. GST-fusion protein of GRF4-PDZ domain binds full-length GRF4 expressed in Hek 293T cells.

FIG. 18. Biotinylated peptide of the last 15 amino acid sequence of GRF4 containing a PDZ-binding motif (SAV*) binds full-length GRF4.

FIG. 19. (a) Nucleic acid molecule sequence [SEQ ID NO: 1] and amino acid sequence [SEQ ID NO: 2]; (b) The figure shows the nucleic acid molecule sequence that is [SEQ ID NO: 3] and amino acid sequences [SEQ ID NOS: 4,5,6]. In a preferred embodiment, [SEQ ID NO: 3] is the Clone 7.7 DNA nucleic acid molecule sequence

FIG. 20. Plasma membrane localization of GRF4.

FIG. 21. GRF4 domain organization and expression. (a) GRF4, depicting its cNMP (cAMP/cGMP) binding domain (cNMP-BD), a Ras Exchange Motif (REM), a PDZ domain, a Ras Association (RA) domain, a CDC25 domain which contains an insert region (white box) and a C terminus which includes 2 PY motifs (PPxY) that bind Nedd4 WW domain(s). The COOH terminus ends with the sequence SAV, conforming to a PDZ binding motif. Sequence alignment of the CDC25, cNMP-BD and PDZ domains is provided in the Supplementary material.

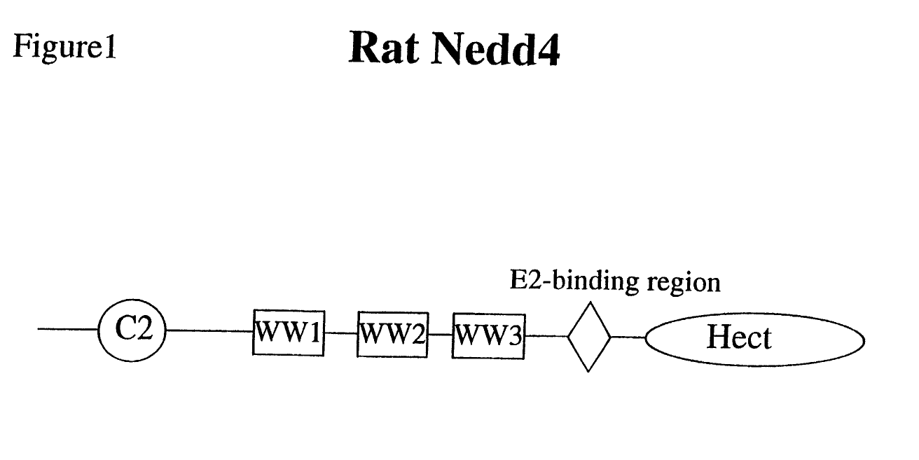

(b) Northern blot analysis of GRF4 mRNA in multiple regions of human brain, probed with the radiolabelled cDNA corresponding to the 3′ region of human GRF4 (nucleotides 4286-4620 of KIAA0313), and depicting expression of ˜7.5 and ˜8.5 kb size transcripts. (blot purchased from Clontech). A multiple rat tissue Northern blot (from Clontech) probed with GRF4 cDNA revealed strong expression primarily in the brain (not shown).

(c) Western blots depicting characterization of anti GRF4 antibodies and expression of the GRF4 protein in synaptosomes. Polyclonal anti GRF4 antibodies were raised against a GST-fusion protein encompassing the C terminus (amino acids 1350-1499) of GRF4, and recognize the ˜180 kDa GRF4 protein either heterologousiy expressed in HEK-293T cells (epitope-tagged with HA, Flag (Fl) or myc tags) (left panel), or endogenously expressed in synaptosomes from adult (Ad) or embryonic (Emb) rat brain (right panel). No protein was detected with the pre-immune (pre-imm) serum. tfxn, transfection; IP, immunoprecipitation; aGRF4, anti GRF4 antibodies.

FIG. 22. Binding of cAMP to the cNMP-binding domain (cNMP-BD) of GRF4.

(a) In vitro binding of GST-GRF4-cNMP-BD, but not GST alone, to immobilized cAMP. cAMP-agarose beads were incubated with soluble GST-GRF4-cNMP-BD or GST alone, washed, proteins separated on 10% SDS-PAGE and immunoblotted with anti GST antibodies (upper panel). Total amount of proteins incubated with the cAMP beads is shown in the lower panel (coomassie).

(b) Precipitation of transfected GRF4, but not mutant GRF4 lacking its CNMP-BD (ΔcNMP-BD), with cAMP agarose beads. cAMP agarose beads were incubated with cell lysates from HEK-293T cells expressing either GRF4 or ΔcNMP-BD, followed by washing of beads, SDS-PAGE, and immunoblotting with anti GRF4 antibodies (upper panels). Expression of full length and mutant GRF4 was verified by immunoblotting aliquots of the respective cell lysates with the same antibodies (bottom panels). Right and left panels in (b) represent two separate experiments.

FIG. 23 cAMP/cGMP-mediated activation of Ras, but not Rap1, by GRF4 in living cells. (a) cAMP-dependent and PKA-independent activation of ras by GRF4.

HEK-293T cells were transfected (or not) with Flag-tagged GRF4, serum-staved overnight, pre-treated (or not) with the PKA inhibitors H-89 (10 μM) or Rp-cAMPS (50 μM) for 30 min., and then treated (or not) with the cAMP analogue 8-Br-cAMP (500 μM) for 15 min. Cells were then lysed and lysate incubated with immobilized Ras binding domain (RBD) of Raf1 (GST-Raf1-RBD), which binds activated (GTP-bound) Ras. Co-precipitated activated ras was then detected with anti Ras antibodies (Quality Biotech) (upper panel). Lower 2 panels depict the amounts of total endogenous Ras and of the transfected GRF4 (detected with anti Ras and anti Flag antibodies, respectively).

(b) cGMP-dependent and PKG-independent activation of ras by GRF4.

Cells were transfected (or not) with Flag-GRF4 and serum-starved overnight as in (a) above, pre-treated (or not) with the PKG inhibitors H8 (5 μM) or Rp-cGMPS (25 μM) and then treated (or not) with the cGMP analogue 8-Br-cGMP (500 μM), as in (a) above. Activated Ras was then precipitated with GST-Raf1-RBD (upper panel), as in (a). Lower panels show total endogenous Ras and GRF4 expressed in the cells.

(c) Activation of Ras via GRF4 following elevation of intracellular levels of cAMP or cGMP.

Cells were transfected as in (a), and were then treated (for 15 min) with either Forskolin (50 μM) plus the cAMP phosphodiesterase inhibitor IBMX (100 μM), to elevate intracellular cAMP, or with YC-1 (100 μM) plus the cGMP phosphodiesterase inhibitor DiPy (10 μM), to elevate intracellular cGMP. Parallel treatments with 8-Br-cAMP or 8-Br-cGMP were used as positive controls. Lysates of treated cells were then incubated with GST-Raf1-RBD to precipitate activated Ras, and immunoblotted with anti Ras antibodies, as above (upper panel). Lower panels are as in (a) and (b) above.

(d) Requirement of the cNMP-BD and the CDC25 domain of GRF4 for cAMP-mediated Ras activation.

HEK-293T cells were transfected with vector alone, GRF4 (WT), GRF4 lacking its cNMP-BD (ΔcNMP-BD) or its CDC25 domain (ΔCDC25), and then treated (or not) with 8-Br-cAMP. Cell lysates were then incubated with GST-Raf1-RBD to precipitate active Ras, and immunobloited with anti Ras antibodies as above (upper panel). Lower panels are controls for total endogenous Ras and transfected GRF4 or its mutants.

(e) In vitro activation of Ras by GRF4.

Full length (GRF4) or CDC25-deleted (ΔCDC25) GRF4, or GRF2, each Flag-tagged, were immunoprecipitated from transfected Hek-293T cells using anti Flag antibodies. Equal amounts of immunoprecipitates were washed with GEF lysis buffer, equilibrated with GEF assay buffer and incubated with 32P-α-GTP (diluted in cold GTP) plus 100 ng of Ras for 30 min. Bound and unbound radiolabelled GTP were then separated by filtration, and the amount of bound GTP determined by scintillation counting, as detailed in the Method section. Fold Ras activation was compared to the activation of Ras in the absence of GRFs (which was set to 1). The number of independent experiments (n), each carried out in duplicates, is indicated in the figure.

Abbreviations: YC-1, 3-(5′-Hydroxymethyl-2′-furyl)-1-benzylindazole; DiPy, Di-Pyridamole; Rp-8-Br-cAMPS, Adenosine 3′,5′-cyclic monophosphorothioate, 8-Bromo-, Rp-isomer; Rp-8-Br-cGMPS, Guanosine 3′,5′-cyclic monophosphorothioate, 8-Bromo-, Rp-isomer; IBMX, 3-isobutol-1-methylxanthine; H-89, N-[2-((Pbromocinnamyi)amino)ethyl]-5-isoquinoiinesulfonamide; H-8, N-[2-(methylamino)ethyl]-5-isoquinolinesulfonamide. Autoradiograms are representative of 2-8 independent experiments.

FIG. 24. GRF4 is localized to the plasma membrane and this localization is dependent on intact PDZ domain but not the SaV C-terminal sequence.

Wild type (WT) GRF4 (panel a), GRF4 lacking the PLPF sequence (-PLPF) of its PDZ domain (equivalent to the hallmark GLGF sequence in numerous PDZ domains) (panel b), or GRF4 in which its final 3 amino acids (SAV), which conform to a PDZ binding motif, were mutated to triple Ala (SAV→AAA)(panel c), were transfected into HEK-293T cells. Transfected cells were fixed and stained with anti GRF4 antibodies followed by FITC-conjugated goat anti rabbit IgG. Images shown represent total cellular fluorescence. Cell diameter˜6 μm.

### DETAILED DESCRIPTION OF THE INVENTION

Identification and characterization of GRF4

The invention includes an isolated Guanine Nucleotide Releasing Factor 4 (GRF4) polypeptide Ras activator. The polypeptide is preferably mammalian, and more preferably human. The invention also includes a recombinant isolated GRF4 protein produced by a cell including a nucleic acid molecule encoding a GRF4 operably linked to a promoter. The invention also includes an isolated nucleic acid molecule encoding a GRF4 polypeptide.

GRF4 was isolated as a PY (xPPxY) motif-containing polypeptide. A 450 nucleotide murine fragment encoding two PY motifs was initially isolated. At the amino acid level this fragment was 75% identical (95% similar) to the hypothetical gene product of the human Genbank entry KIAA0313. We characterized the human polypeptide, which we named GRF4 (also known as RasGRF4), because it is a fourth class of Ras guanine nucleotide exchange factor (GEF). GRF4 polypeptides were unknown prior to this invention. The hypothetical polypeptide based on KIAA0313 DNA sequence information cannot predict if a polypeptide is translated, its sequence, activity or the extent of post-translational modifications.

The invention includes GRF4 nucleic acid molecules and molecules having sequence identity or which hybridize to the GRF4 sequence which encode a protein capable of activating Ras (preferred percentages for sequence identity are described below) as well as vectors including these molecules. The invention also includes GRF4 or proteins having sequence identity (preferred percentages described below) or which are capable of activating Ras. The nucleic acid molecules and proteins of the invention may be from lung, brain or the neuronal system and they may be isolated from a native source, synthetic or recombinant. The invention includes GRF4 or proteins having sequence identity which are capable of activating Ras, as prepared by the processes described in this application.

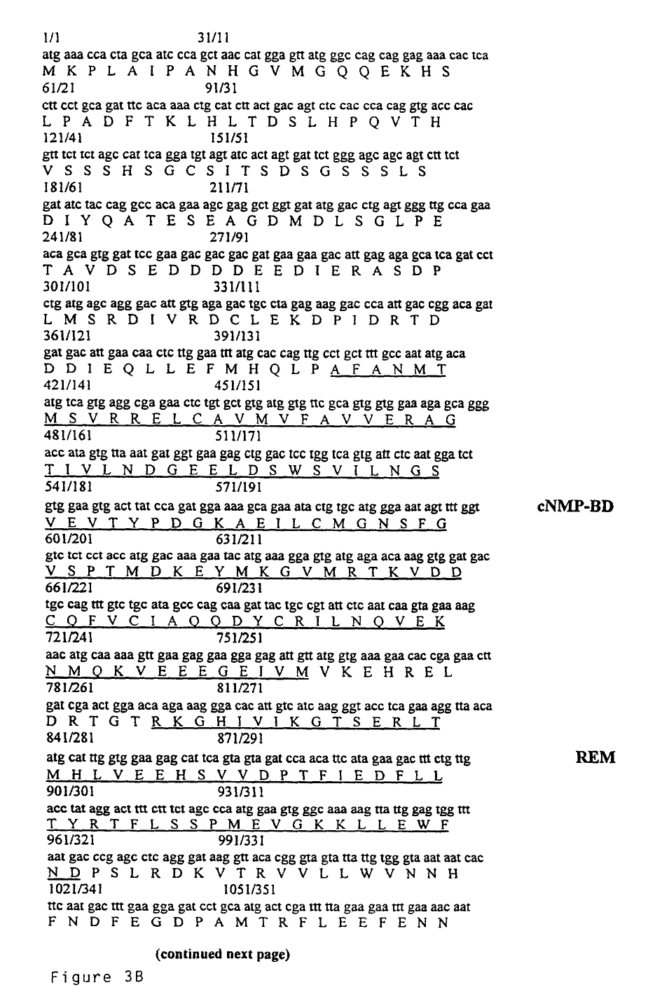

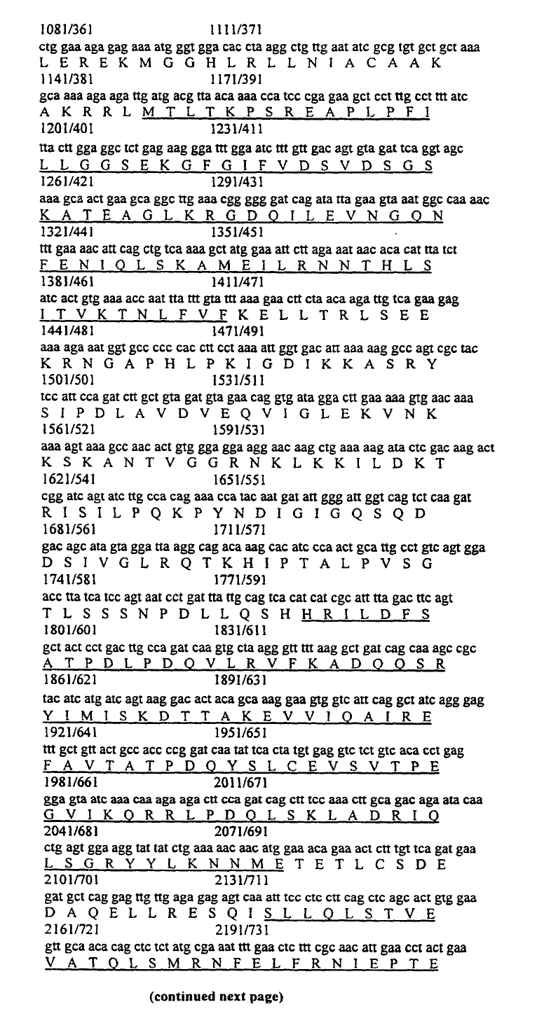

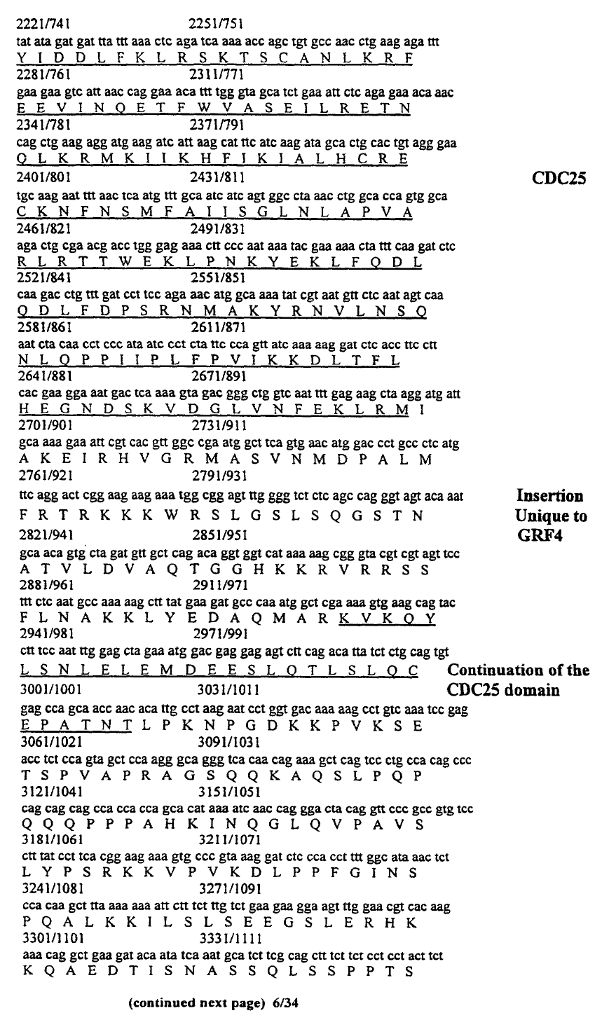

This GRF represents a fourth class of RasGRFs. FIG. 3 is a schematic diagram of GRF4. The structural features of GRF4 show a multifunctional role that regulates several aspects of cell physiology, including cell proliferation, morphology, membrane transport, cell survival and cellular transformation. GRF4 expression, concentration and activity may be manipulated in methods of medical treatment of excessive cell proliferation, such as in cancer (for example, brain cancer, lung cancer).

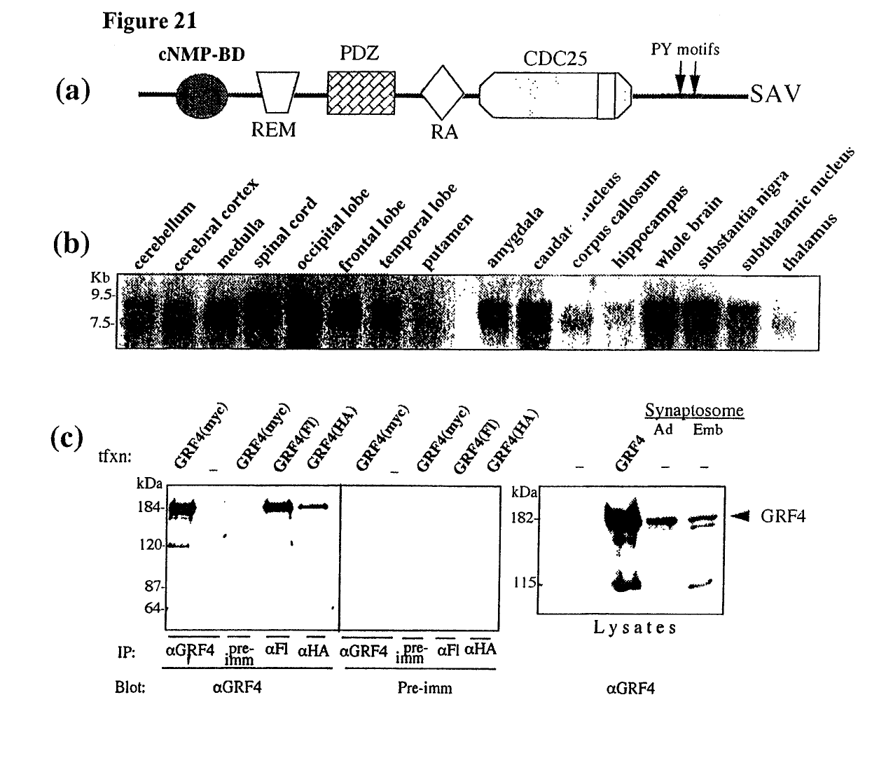

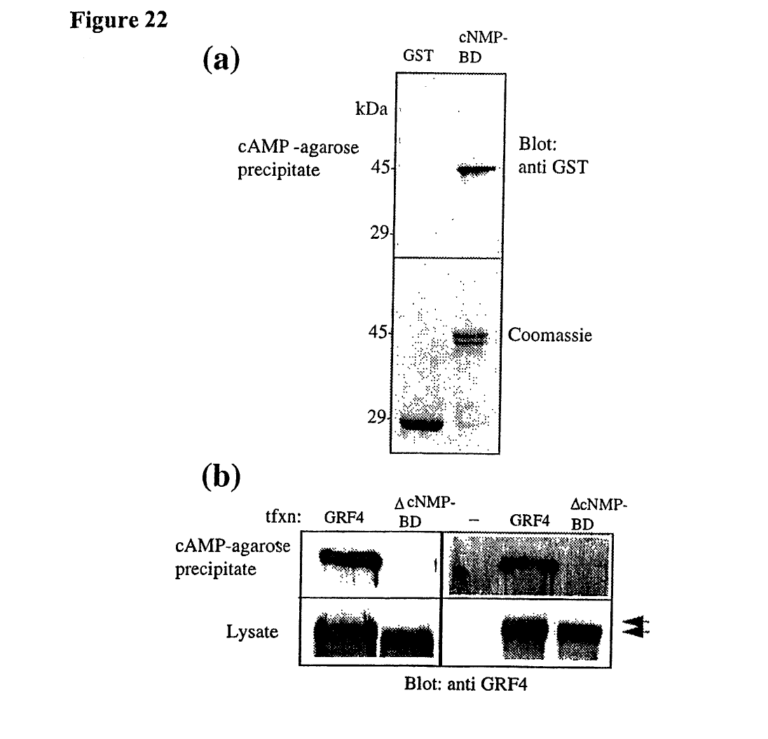

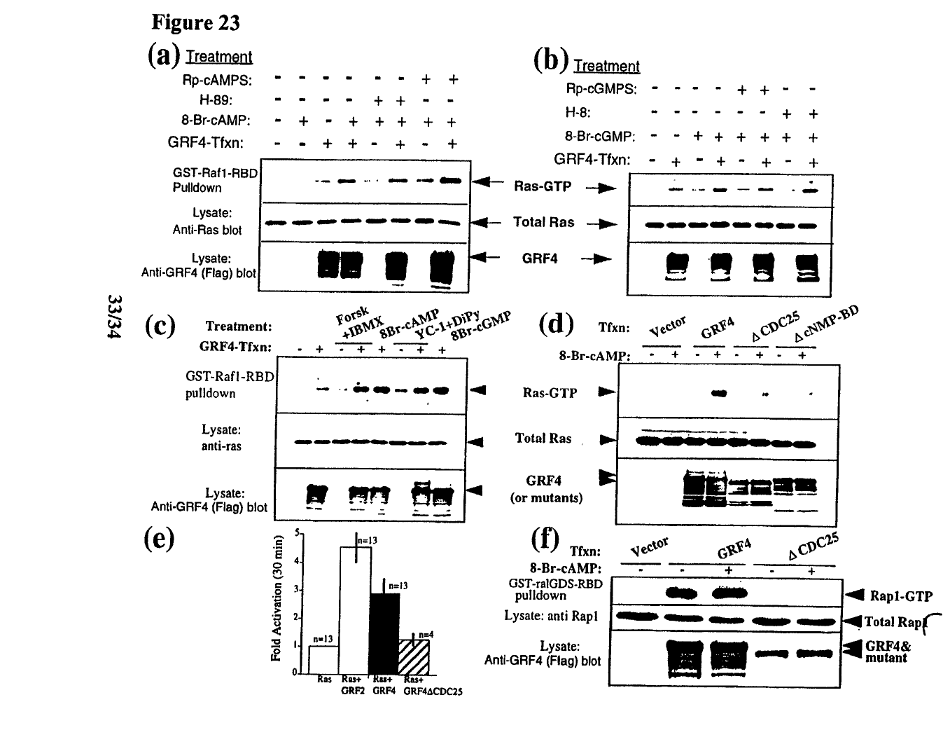

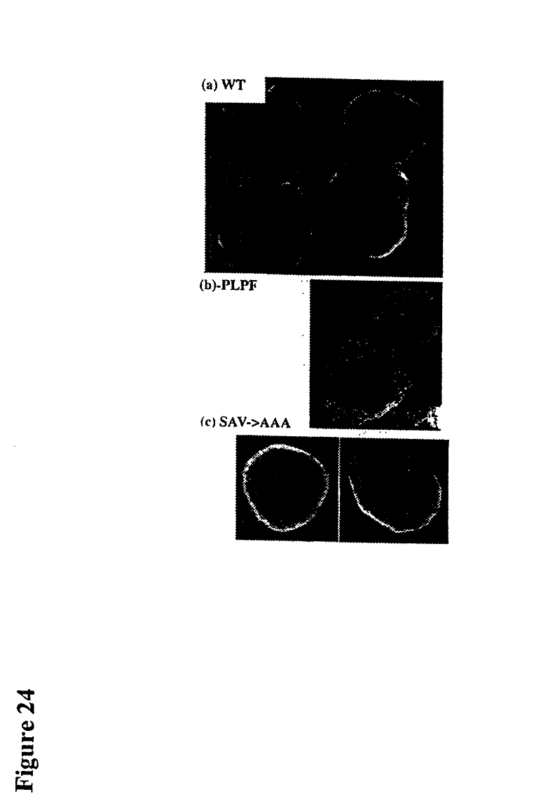

GRF4 is composed of several recognizable sequence motifs and domains. GRF4 contains, in amino to carboxyl order, a cyclic nucleotide monophosphate (cAMP/cGMP)-Binding domain (cNMP-BD), a Ras exchange motif (REM), PDZ and Ras association (RA) domains, CDC25-related GEF domain, two PY motifs responsible for binding to the Nedd4-WW domain, and a COOH-terminal SaV sequence conforming to PDZ binding motif. The Cdc25 domain is similar to the Ras GEF regions in Sos½, GRF½ and GRP, as well as in Rap GEFs such as Epac (H. Kawasaki, et al., 1998; J. de Rooij, et al., 1998). The GRF4-Cdc25 domain contains a unique insertion, located on the carboxyl side of the third structurally conserved region (SCR3; see alignment) (P. A. Boriack-Sjodin et al., 1998). The PDZ domain of GRF4 appears most similar to the PDZ domains of Lin7, PTP-BAS, PSD-95 and Dig (see alignment). PDZ domains have been shown to be involved in intracellular targeting and clustering of plasma membrane proteins and signaling complexes (S. E. Craven et al., 1998). The cNMP-BD of GRF4 is similar to the cAMP binding region of protein kinase A (PKA) and cAMP-GEFs (H. Kawasaki, et al., 1998; J. de Rooij, et al., 1998) and to the cGMP binding region of protein kinase G (PKG) and cyclic nucleotide gated K⁺ channels although it lacks the conserved RAA motif found in PKA and cAMP-GEFs, and the conserved RTA motif found in PKG and cyclic nucleotide gated K⁺ channel (see alignment). These conserved motifs were shown to play a role in conferring specificity for binding of cAMP or cGMP, respectively (Y. Su, et al., 1998). GRF4 mRNA is expressed predominantly in the brain, with widespread distribution (FIG. 21b). Accordingly, the ˜180 kDa GRF4 protein is detected in the brain, including embryonic and adult rat brain synaptosomes, but not in several fibroblasts cell lines, or the human epithelial (embryonic kidney) cell line HEK-293T (FIG. 21c).

GRF4 activity and effects on Ras and Rap1

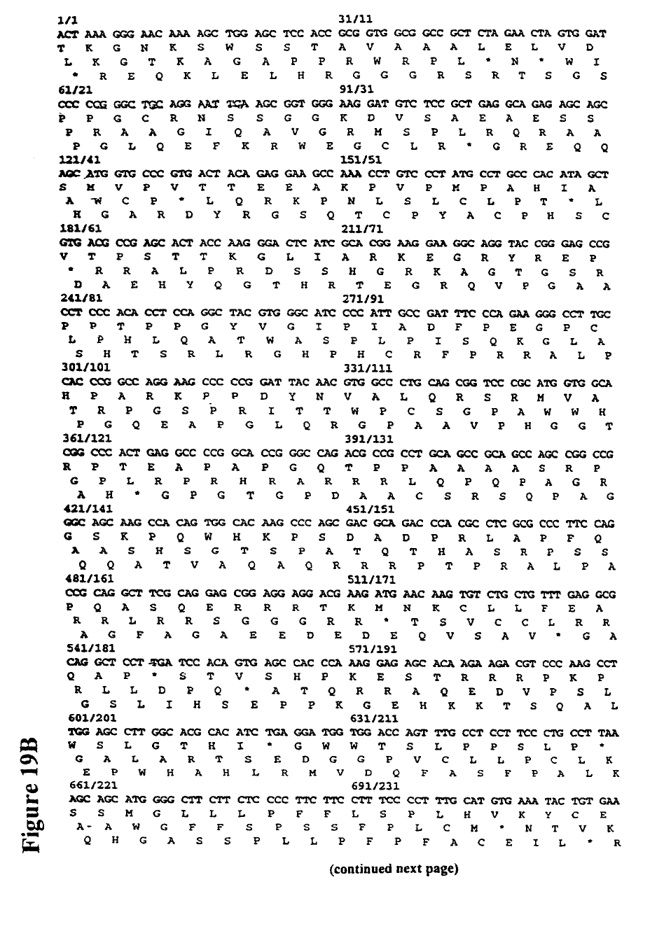

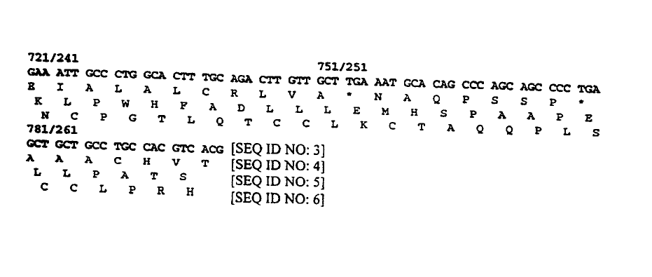

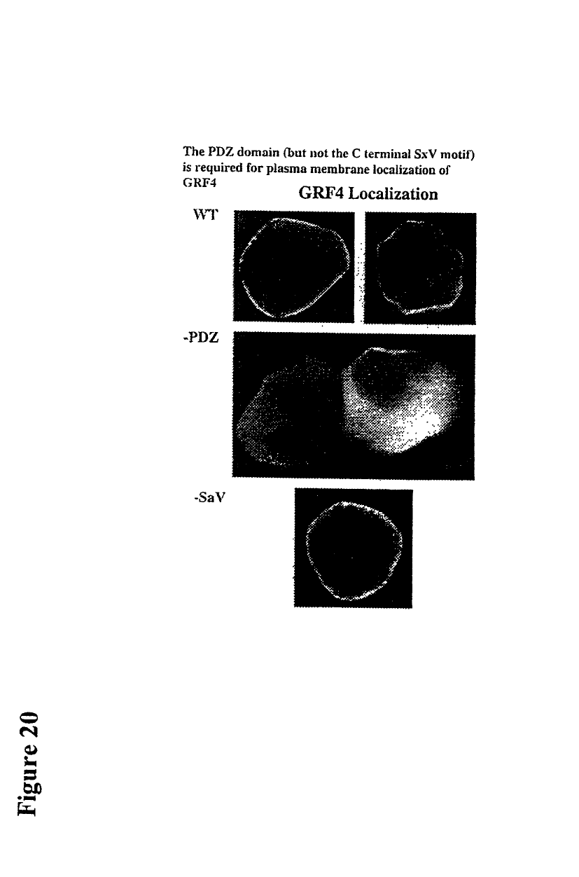

GRF4 is activated by distinct signaling pathways that involve a G-coupled receptor signaling pathway (FIG. 19). GRF4 can be activated by a G-protein coupled receptor via an association of GRF4-PDZ domain and its binding motif present in many such receptor. This activation process depends on the activation state of the receptor. Binding of GRF4 to such a receptor leads to activation of GRF4 as a result of conformational changes or membrane recruitment of GRF4 (or both). In one of the aspects of the inventions, activation of a G-coupled receptor leads to elevation of cAMP which modulates GRF4 activity by directly binding to GRF4-cAMP-BD. The SAV* motif of GRF4 can be involved in an intramolecular interaction with GRF4-PDZ domain and this interaction may have regulatory roles in GRF4 activity. Likewise, this motif can bind to other PDZ-containing proteins associating with the plasma membrane. GRF4 binds preferentially to nucleotide-free and GTP-bound Ras. The RA domain of GRF4 mediates GRF4 binding to Ras-GTP. In so doing, GRF4 functions as a downstream Ras effector. The ubiquitin protein ligase Nedd4 interacts with GRF4 through WW domain-PY motif interaction and ubiquitinates GRF4 and targets it for degradation.

Ras and Rap1 have distinct subcellular localizations and interact with an overlapping set of effector proteins and signaling pathways (reviewed in J. L. Bos, 1998). GRF4 activates both Rap1 and Ras, and the activation of Ras, but not Rap1, is stimulated by cAMP or cGMP binding to GRF4. Elevation of intracellular cAMP or cGMP levels causes a switch from Rap1 to Ras activation by GRF4. In view of its strong expression in brain and synaptosomes, activation by cyclic nucleotides, and presence of a PDZ domain, a plasma membrane protein such as a G protein-coupled receptor which causes elevation of cAMP upon activation, or ion channels enriched in synaptosomes (several of which possess a PDZ-binding motif, are activators of GRF4. The presence of GRF4 in the vicinity or even in complex with such proteins shows a direct connection between them and Ras/Rap1 activation. GRF4 was identified in this study as a Nedd4-interacting protein, and our recent work has detected Nedd4 at the plasma membrane and in endosomes. The internalization of plasma membrane-associated GRF4, possibly in complex with its cell surface activator and/or Nedd4, provides a mechanism to regulate GRF4 interactions with Ras at the plasma membrane and Rapi in the endocytic compartment. The association with Nedd4 regulates of stability of GRF4 (or associated proteins) by ubiquitination.

GRF4 domains and motifs

The table below shows the amino acid sequence number (in bracket) and the nucleotide sequence (in square bracket) for the domains and motifs shown in the GRF sequence.

TABLE 1

| | | |
|---|---|---|
| Full length GRF4: | (1-1499), | [1-4500]-the nucleotide sequence |
| | | includes the Stop codon |
| | | (FIG. 3(b)). |
| cNMP-BD: | (135-253), | [403-759] |
| REM: | (266-322), | [796-966] |
| PDZ: | (386-470), | [1156-1410] |
| RA: | (594-692), | [1780-2076] |
| CDC25: | (712-1006), | [2134-3018] |
| Insertion in CDC25: | (900-975), | [2697-2925] |
| First PY motif: | (1403-1406), | [4207-4218] |
| Second PY motif: | (1425-1428), | [4273-4284] |
| SAV motif: | (1497-1499), | [4489-4497] |

PY-motifs

GRF4 contains two PY-motifs near the C-terminus which bind to Nedd4-WW domains leading to its identification as a Nedd4-WW domain interacting protein in the expression library screen. Preferable protein hybridization conditions use TBS-Tween (about: 137 mM NaCl, 27 mM KCl, 25 mM tris, pH 8.0, 0.1% Tween 20). The screen used to identify Clone 7.7 was based on protein:protein interactions (i.e. a labeled GST Nedd4-WW domain protein was used as a probe to screen an expression library. cDNA of the library was induced to express proteins. Washes were done with TBS-Tween). These conditions can be used in a method to identify other GRF proteins similar to GRF4 which preferably have GRF4 activity or similar activity.

CDC25 Domains

GRF4 harbours a central catalytic region called CDC25 domain, named for the prototypic Ras activator in Saccharomyces cerevisiae (21), from which the function of GRF4 was deduced.

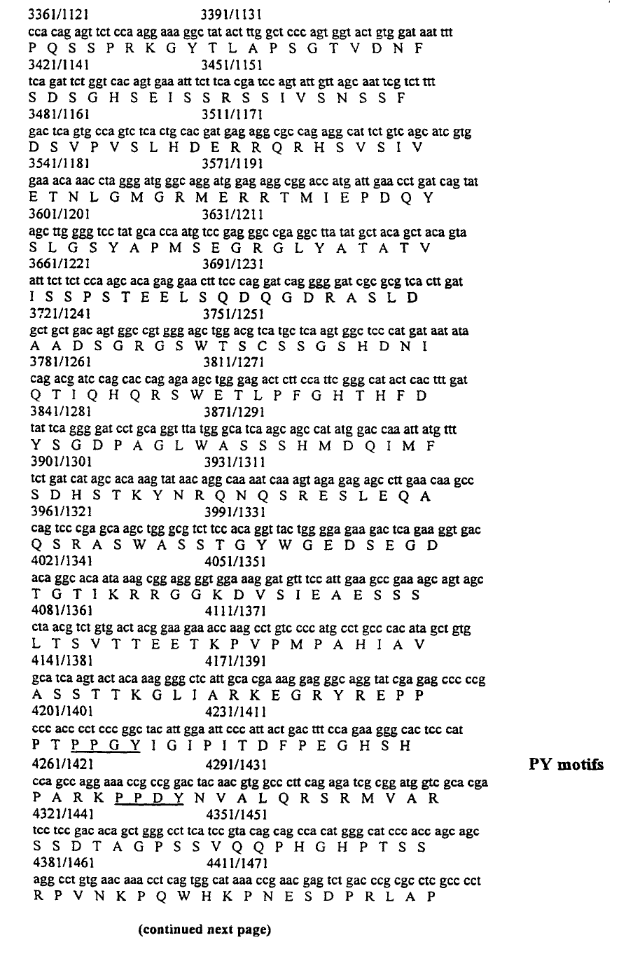

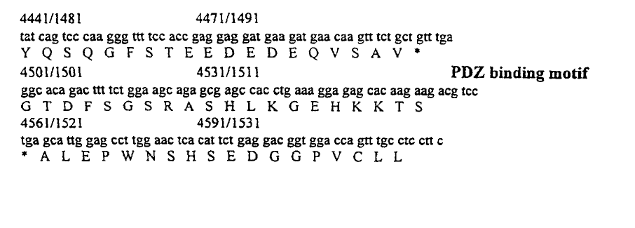

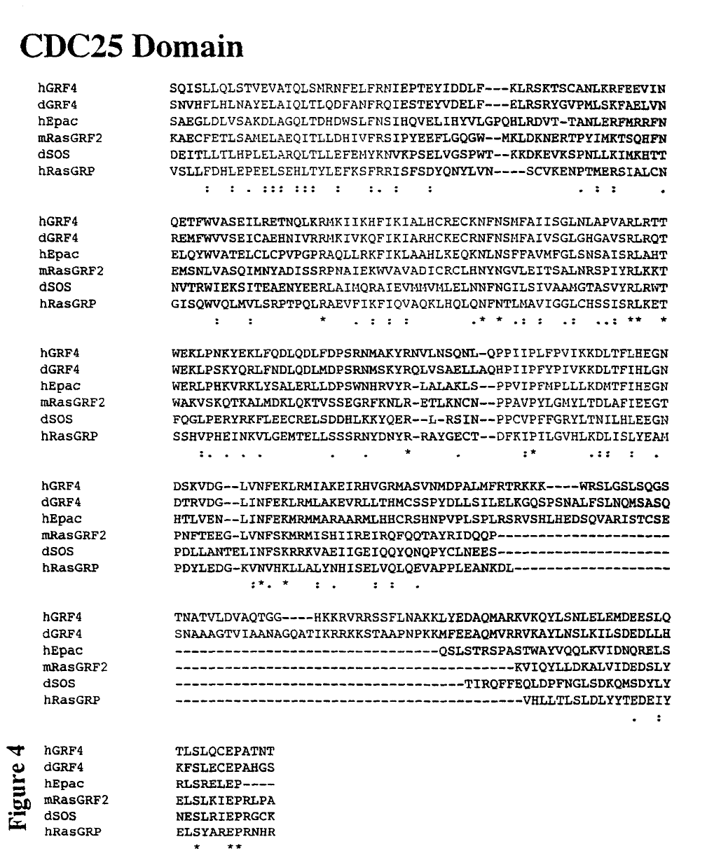

CDC25 domains catalyze guanine-nucleotide exchange/release activity on Ras family GTPases. The CDC25 of GRF4 is 48-52% similar to those of yeast CDC25, SOS and RasGRF/RasGRF2. FIG. 4 shows the alignment of CDC25 domains from various proteins including GRF4. From the mutagenesis studies of yeast CDC25, several conserved arginine residues were proposed to be critical for its activity (22). These conserved arginine residues are also conserved in GRF4. Similar to CDC25, SDC25, RasGRF½ and SOS, GRF4 contains blocks of highly conserved sequences (FIG. 4A) which were recently demonstrated, based on the tertiary structure of SOS bound to Ras, to play a critical role in the activity of the CDC25 domain towards Ras (23). However, unique to GRF4, the GRF4-CDC25 domain also contains an insert (about 40 amino acids) not found in SOS, RasGRF2 or other RasGRF3 (FIG. 3B).

Ras Exchange of Motif Domain

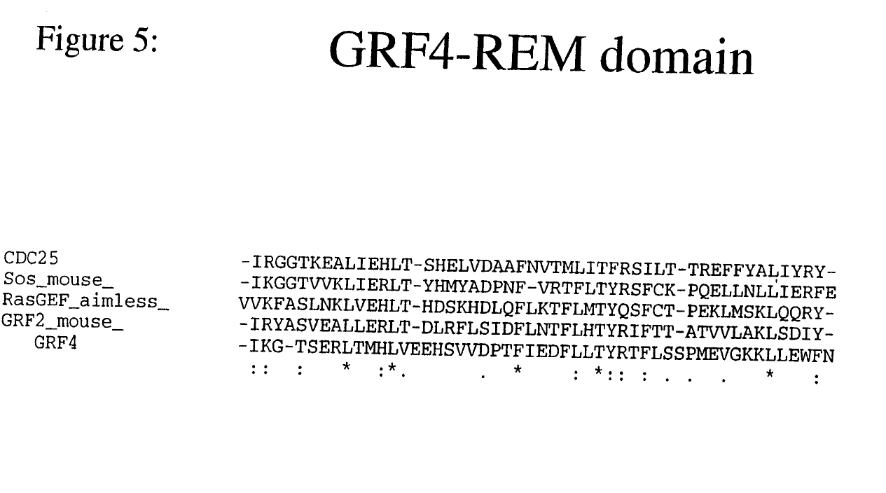

GRF4 also has a REM (Ras exchange motif) domain (24) which is present in all known mammalian RasGRFs. FIG. 5 shows the alignment of REM domains from several proteins including GRF4. Mammalian RasGRFs all share this REM domain which is likely important for their activities. Recently, it was reported that the REM domain of SOS contributes to the activity of the CDC25 domain by stabilizing the active structure of the catalytic region (23).

Diacylqlycerol Binding Domain, EF Hands, Calcium Binding Motif

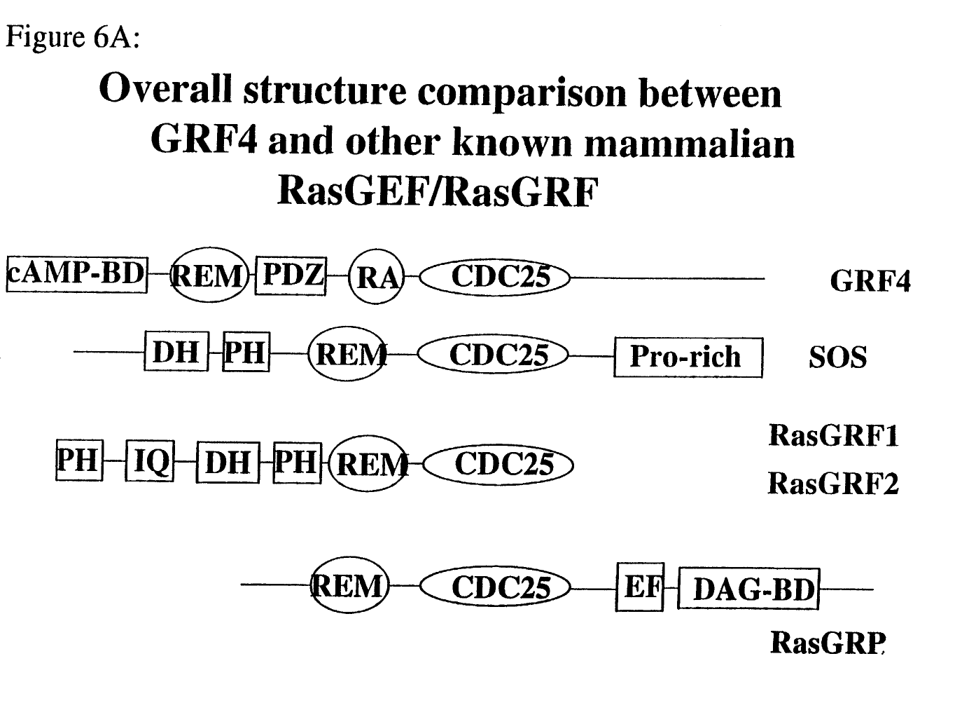

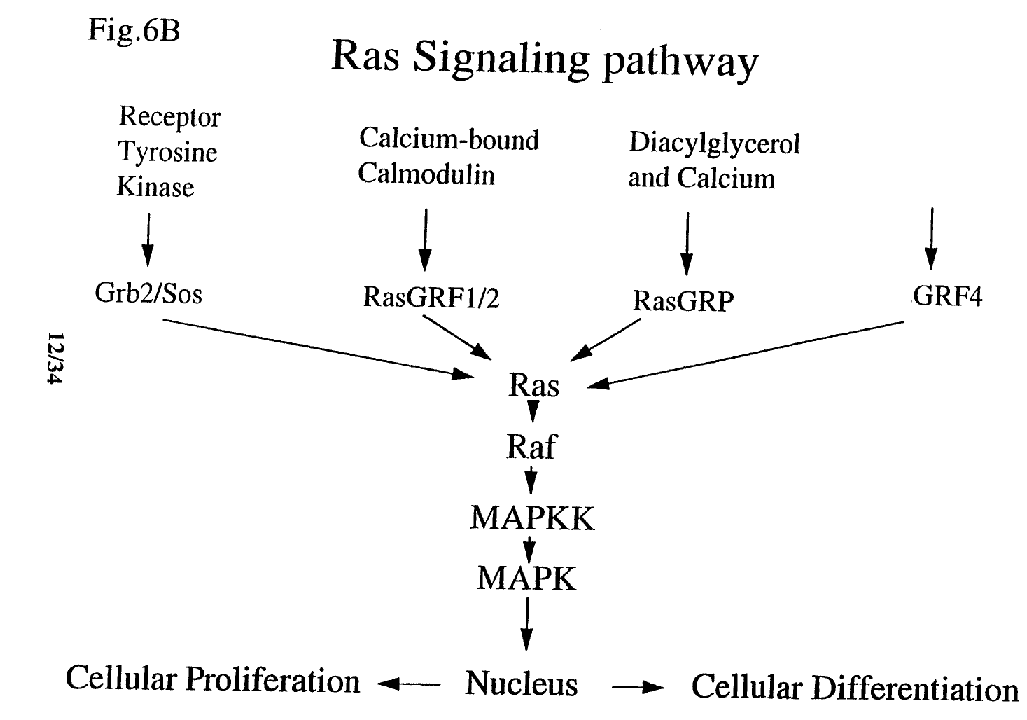

As shown in FIG. 6A, each mammalian RasGRF has its own unique domains which are important for regulation of its activity. Specifically, SOS is activated by various growth factors, a process involving binding of activated receptor-tyrosine kinase to Grb2-SH2 domain and Grb2-SH3 domain to the proline-rich region of SOS-(25). RasGRF1 and RasGRF2 are activated by elevation of intracellular calcium, a process involving the binding of Ca2+-bound calmodulin to the IQ motif present in these RasGRFs (23, 26). RasGRP harbours a DAG (diacylglycerol) binding domain and a pair of EF hands, a Ca2+ binding motif and accordingly, it is activated by elevated level of DAG and calcium (27). These unique domains allow RasGRFs to activate Ras in response to distinct signaling pathways. The small GTPase Ras controls the MAPK pathway, (as well as PI-3 kinase, raIGEF and likely other effectors). In so doing, Ras exerts its effects on many cellular processes such as cellular proliferation and differentiation (FIG. 6B).

PDZ Domains

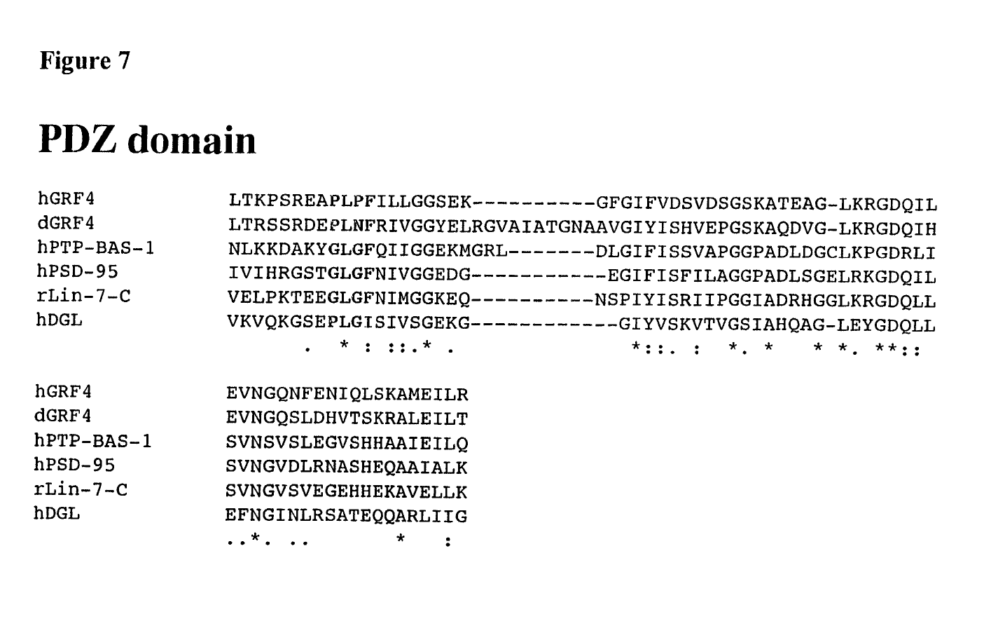

PDZ (PSD95/DIg/ZO-1) domains, also known as DHR (Disc-large homology region) or GLGF domains (conserved stretch of amino acids in the domain) are 80-100 amino acid protein-protein interaction modules which are found in membrane-associating proteins and intracellular signaling proteins (Ref. 28). PDZ domains are important for membrane targeting, clustering of receptors/channels and forming scaffold of networks of signaling proteins at the plasma membrane. Examples include PSD-95 which binds the NMDA receptors, as well as the InaD which binds to the TRP, components of photo-transduction cascades in the Drosophila eyes (29-30). PDZ domains bind to C-terminal three or four residues in a sequence specific context. One class of PDZ domains, including those of Disc-large protein, binds to C-terminal Valine residue in a context of S/T×V* (* denotes a stop codon). While other classes of PDZ domains were shown to bind C-terminal three residues with hydrophobic or aromatic side chains (31). The alignment of PDZ domains of several proteins including GRF4 is given in FIG. 7. The PDZ domain of GRF4 is similar to a class of PDZ domains binding S/T×V* motif. GRF4 itself has such a motif (SAV*) at its C-terminus (FIG. 3), so there is interaction between GRF4-PDZ domain and its own PDZ-binding motif.

cNMP Binding Domain

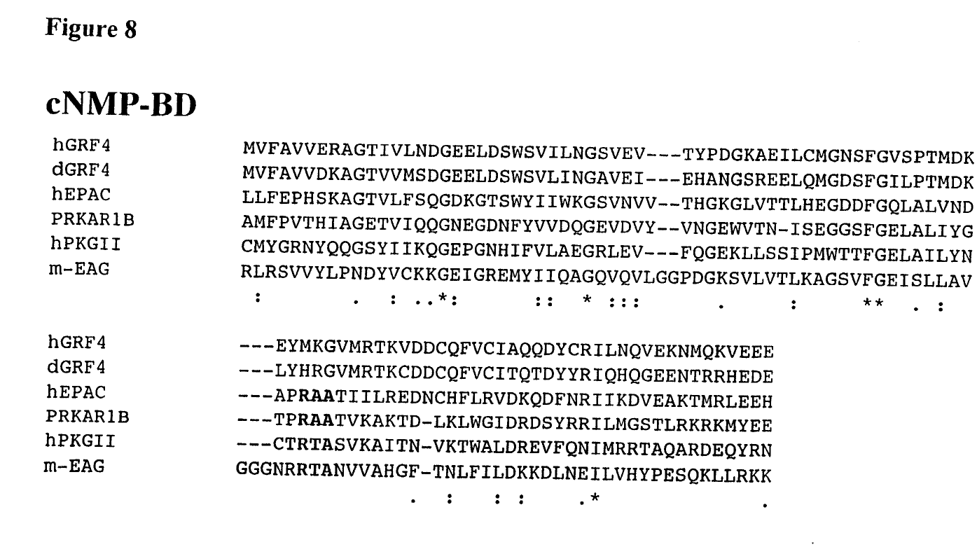

GRF4 has a cNMP-binding domain that preferably binds cAMP or cGMP. It shares 50% sequence similarity to that of the regulatory subunits of PKA. FIG. 8 shows the alignment of cNMP-binding domains. Since a conformational change is often accompanied by binding of cNMP to a protein, GRF4 activity may be regulated by conformational changes. By having a cAMP-binding domain, GRF4 is involved in a G-coupled receptor pathway and connects this pathway to the Ras signaling pathway. Many G-protein coupled receptors contain PDZ-binding motifs which bind and regulate activities of PDZ- domain containing proteins. Having both a PDZ domain and a cAMP binding domain, GRF4 is regulated by a G-coupled receptor system coupling to the adenylyl cyclase enzyme. Altematively, when cGMP is the compound binding and activating (or inhibiting) GRF4, RasGRF directly connects upstream activators of cGMP release (e.g. nitric oxide) to Ras.

Ras Associating Domain

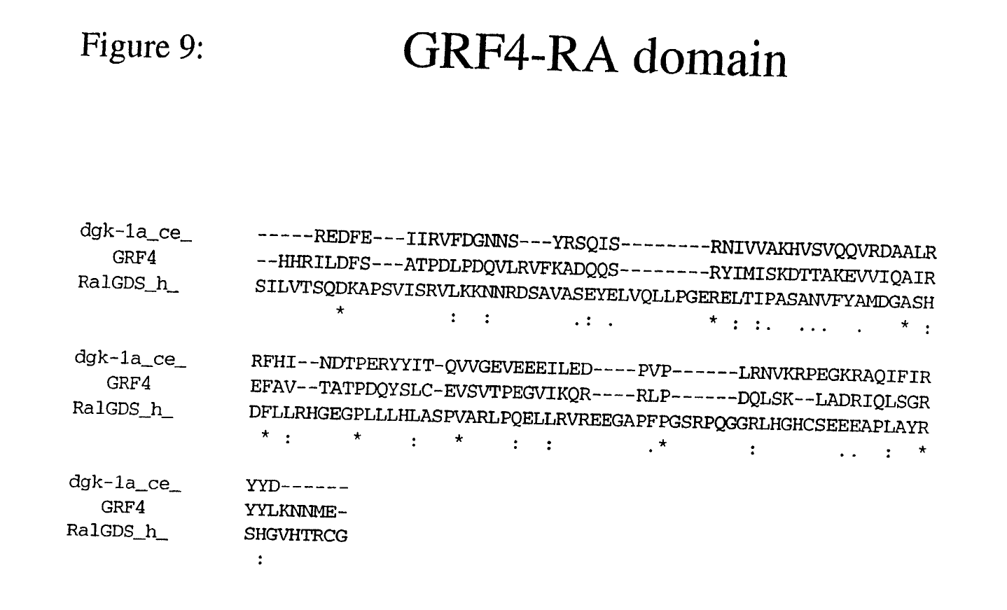

GRF4 also has a RA (Ras associating) domain. This type of domain was initially identified in two Ras effector proteins, including RaIGDS and AF-6/Canoe, and later in numerous Ras binding proteins. RA domains have been assumed to bind to Ras-GTP and the solved tertiary structure of RaIGDS-RA domain was found to be similar to that of the Ras binding domain of Raf kinase which binds to Ras-GTP (32). However, recent evidence shows that not all RA domains bind to Ras-GTP. The alignment of RA domains from several proteins including GRF4 is given in FIG. 9.

PEST Sequences, coil-coil and PY motifs

In addition to the above domains, GRF4 has two PEST sequences which are after found in unstable proteins. GRF4 also has a coiled-coil region which participates in protein-protein interaction through interactions of multiple amphipathic alpha helices (33). The PY motifs serve as attachment sites for the Nedd4-WW domain, thereby facilitating ubiquitination and degradation of GRF4.

Functionally equivalent nucleic acid molecules

The invention includes nucleic acid molecules that are functional equivalents of all or part of the sequence in [SEQ ID NO: 1]. (A nucleic acid molecule may also be referred to as a DNA sequence or nucleotide sequence in this application. All these terms have the same meaning as nucleic acid molecule and may be used to refer, for example, to a cDNA, complete gene or a gene fragment. The intended meaning will be clear to a person skilled in the art.) Functionally equivalent nucleic acid molecules are DNA and RNA (such as genomic DNA, cDNA, synthetic DNA, and mRNA nucleic acid molecules), that encode peptides, proteins, and polypeptides having the same or similar GRF4 activity as the GRF4 polypeptide shown in (SEQ ID NO: 23. Functionally equivalent nucleic acid molecules can encode peptides, polypeptides and proteins that contain a region having sequence identity to a region of a GRF4 polypeptide or more preferably to the entire GRF4 polypeptide. The CDC25 is a preferred region because it is the central catalytic region. The invention includes nucleic acid molecules that have a region with sequence identity to the CDC25 coding region of [SEQ ID NO: 1] which is represented by about nucleotide no. 2194 (2131+63) to nucleotide no. 3082 (preferred percentages of identity are below). The invention includes nucleic acid molecules about: <1000 nucleotides (preferably about 888 nucleotides), <1500 nucleotides, <2000 nucleotides, <3000 nucleotides or <5000 nucleotides which encode a region having sequence identity to the CDC25 coding region and having CDC25 activity or CDC25-ike activity.

Identity is calculated according to methods known in the art. The Clustal W program (preferably using default parameters) [Thompson, J D et al., Nucleic Acid Res. 22:4673-4680.], described below, is most preferred. For example, if a nucleic acid molecule (called “Sequence A”) has 90% identity to a portion of the nucleic acid molecule in [SEQ ID NO: 1], then Sequence A will preferably be identical to the referenced portion of the nucleic acid molecule in [SEQ ID NO: 1], except that Sequence A may include up to 10 point mutations, such as substitutions with other nucleotides, per each 100 amino acids of the referenced portion of the nucleic acid molecule in [SEQ ID NO: 1]. Mutations described in this application preferably do not disrupt the reading frame of the coding sequence. Nucleic acid molecules functionally equivalent to the GRF4 sequences can occur in a variety of forms as described below.

Nucleic acid molecules may encode conservative amino acid changes in GRF4 polypeptide. The invention includes functionally equivalent nucleic acid molecules that encode conservative amino acid changes within a GRF4 amino acid sequence and produce silent amino acid changes in GRF4.

Nucleic acid molecules may encode non-conservative amino acid substitutions, additions or deletions in GRF4 polypeptide. The invention includes functionally equivalent nucleic acid molecules that make non conservative amino acid changes within the GRF4 amino acid sequence in [SEQ ID NO: 2]. Functionally equivalent nucleic acid molecules include DNA and RNA that encode peptides, polypeptides and proteins having non-conservative amino acid substitutions (preferably substitution of a chemically similar amino acid), additions, or deletions but which also retain the same or similar GRF4 activity as the GRF4 polypeptide shown in [SEQ ID NO: 2]. The DNA or RNA can encode fragments or variants of GRF4. Fragments are useful as imminogens and in immunogenic compositions (U.S. Pat. No. 5,837,472). The GRF4 or GRF4 -like activity of such fragments and variants is identified by assays as described below. Fragments and variants of GRF4 encompassed by the present invention should preferably have at least about 40%, 60%, 80% or 95% sequence identity or preferably at least about 96%, 97%, 98%, 99%, 99.5%, 99.9% or more preferably at least about 99.95% sequence identity to the naturally occurring GRF4 nucleic acid molecule (preferably measured between the coding region of the sequence nucleotides 63 to 4562), or a region of the sequence, such as the coding sequence or one of the conserved domains of the nucleic acid molecule, without being identical to the sequence in [SEQ ID NO: 1]. These sequences preferably encode all the GRF4 domains and motifs described above. One or more domain or motif may be omitted to obtain desired activity. The CDC25 domain is preferably conserved in the nucleic acid molecule and polypeptide in order to preserve GRF4 activity. Sequence identity is preferably measured with the Clustal W program (preferably using default parameters) [Thompson, J D et al., Nucleic Acid Res. 22:4673-4680.]. In another embodiment, the Gap program may be used. The algorithm of Needleman and Wunsch (1970 J. Mol. Biol. 48:443-453) is used in the Gap program. BestFit may also be used to measure sequence identity. It aligns the best segment of similarity between two sequences. Alignments are made using the local homology algorithm of Smith and Waterman (1981) Adv. Appl. Math. 2:482-489. Most preferably, 1, 2, 3, 4, 5, 5-10, 10-15, 15-25, 25-50, 50-100 or 100-600 nucleotides are modified. One would be able to make more changes to the nucleotide and amino acid sequences (such as substitutions, deletions) in regions outside of the conserved regions of GRF4 described above.

Nucleic acid molecules functionally equivalent to the GRF4 in [SEQ ID NO: 1] will be apparent from the following description. For example, the sequence shown in [SEQ ID NO: 1] may have its length altered by natural or artificial mutations such as partial nucleotide insertion or deletion, so that when the entire length of the coding sequence within [SEQ ID NO: 1], is taken as 100%, the functional equivalent nucleic acid molecule preferably has a length of about 60-120% thereof, more preferably about 80-110% thereof. Fragments may be less than 60%.

Nucleic acid molecules containing partial (usually 80% or less, preferably 60% or less, more preferably 40% or less of the entire length) natural or artificial mutations so that some codons in these sequences code for different amino acids, but wherein the resulting polypeptide retains the same or similar GRF4 activity as that of a naturally occurring GRF4 polypeptide. The mutated DNAs created in this manner should preferably encode a polypeptide having at least about 40%, preferably at least about 60%, at least about 80%, and more preferably at least about 90% or 95%, and most preferably at least about 97%, 98%, 99%, 99.5%, 99.9%, or 99.95% sequence identity to the amino acid sequence of the GRF4 polypeptide in [SEQ ID NO: 2]. Sequence identity is preferably assessed by the Clustal W program.

Since the genetic code is degenerate, the nucleic acid sequence in [SEQ ID NO: 1] is not the only sequence which may code for a polypeptide having GRF4 activity. This invention includes nucleic acid molecules that have the same essential genetic information as the nucleic acid molecule described in [SEQ ID NO: 1] or a domain or motif of this region. Nucleic acid molecules (including RNA) having one or more nucleic acid changes compared to the sequences described in this application and which result in production of a polypeptide shown in [SEQ ID NO: 2] are within the scope of the invention.

Other functional equivalent forms of GRF4-encoding nucleic acids can be isolated using conventional DNA-DNA or DNA-RNA hybridization techniques. Thus, the present invention also includes nucleic acid molecules that hybridize to one or more of the sequences in [SEQ ID NO: 1] or its complementary sequence, and that encode expression for peptides, polypeptides and proteins exhibiting the same or similar activity as that of the GRF4 polypeptide produced by the DNA in [SEQ ID NO: 1] or its variants. Such nucleic acid molecules preferably hybridize to the sequence in [SEQ ID NO: 1] under moderate to high stringency conditions (see Sambrook et al. Molecular Cloning: A Laboratory Manual, Most Recent Edition, Cold Spring Harbor Laboratory Press, Cold Spring Harbor, N.Y.). High stringency washes have low salt (preferably about 0.2% SSC), and low stringency washes have high salt (preferably about 2% SSC). A temperature of about 37° C. or about 42° C. is considered low stringency, and a temperature of about 50-65° C. is high stringency. The invention also includes a method of identifying nucleic acid molecules encoding a GRF4 activator polypeptide (preferably a mammalian polypeptide), including contacting a sample containing nucleic acid molecules including all or part of [SEQ ID NO: 1] (preferably at least about 15 or 30 nucleotides of [SEQ ID NO: 1]) under moderate or high stringency hybridization conditions and identifying nucleic acid molecules which hybridize to the nucleic acid molecules including all or part of [SEQ ID NO: 1].). [SEQ ID NO: 3] may be used in a similar manner. Similar methods are described in U.S. Pat. No. 5,851,788 which is incorporated by reference in its entirety.

The invention also includes methods of using all or part of the nucleic acid molecules which hybridize to all or part of [SEQ ID NO: 1 or 3], for example as probes or in assays to identify antagonists or inhibitors of the polypeptides produced by the nucleic acid molecules (described below). The invention also includes methods of using nucleic acid molecules having sequence identity to the GRF4 nucleic acid molecule (as described below) in similar methods. Polypeptides based on all or part of [SEQ ID NOS: 2, 4, 5, or 6] are also useful as probes.

The invention also includes a nucleic acid molecule detection kit including, preferably in a suitable container means or attached to a surface, a nucleic acid molecule of the invention encoding GRF4 or a polypeptide having GRF4 activity and a detection reagent (such as a detectable label). Other variants of kits will be apparent from this description and teachings in patents such as U.S. Pat. Nos. 5,837,472 and 5,801,233 which are Incorporated by reference in their entirety.

For example, Hybridization solution 1 is low stringency: about: >50% formamide, >5×denhardt's, >1% SDS, >5×SSC, >42° C.; Hybridization solution 2 is high stringency: about: >1% BSA, >1 mM EDTA, >0.5 M NaHPO4, pH 7.2, >7% SDS, >65° C. A preferable high stringency wash consists of about: >0.2×SSC, >0.1% SDS. A preferable low stringency wash has about: >2×SSC, >0.1% SDS). These conditions may be varied as known in the art. The present invention also includes nucleic acid molecules that hybridize to genomic DNA, cDNA, or synthetic DNA molecules that encode the amino acid sequence of the GRF4 polypeptide, or genetically degenerate forms, under salt and temperature conditions equivalent to those described in this application, and that encode a peptide, polypeptide or polypeptide that has the same or similar activity as the GRF4 polypeptide. In a preferred embodiment, the invention includes DNA that hybridizes to all or part of the CDC25 coding region of [SEQ ID NO: 1] which is represented by about nucleotide no. 2194 (2131+63) to nucleotide no. 3082, under moderate to high stringency conditions.

A nucleic acid molecule described above is considered to have a function substantially equivalent to the GRF4 nucleic acid molecules of the present invention if the polypeptide produced by the nucleic acid molecule has GRF4 activity. A polypeptide has GRF4 activity if it can activate Ras. Activation of Ras is shown where a polypeptide is active in catalyzing guanine-nucleotide exchange on small GTPase Ras using the in vitro GEF assay.

Production of GRF4 in eukaryotic and prokaryotic cells

The nucleic acid molecules of the invention may be obtained from a cDNA library. The nucleotide molecules can also be obtained from other sources known in the art such as expressed sequence tag analysis or in vitro synthesis. The DNA described in this application (including variants that are functional equivalents) can be introduced into and expressed in a variety of eukaryotic and prokaryotic host cells. A recombinant nucleic acid molecule for the GRF4 contains suitable operatively linked transcriptional or translational regulatory elements. Suitable regulatory elements are derived from a variety of sources, and they may be readily selected by one with ordinary skill in the art (Sambrook, J, Fritsch, E. E. & Maniatis, T. (Most Recent Edition). Molecular Cloning: A laboratory manual. Cold Spring Harbor Laboratory Press. New York; Ausubel et al. (Most Recent Edition) Current Protocols in Molecular Biology, John Wiley & Sons, Inc.). For example, if one were to upregulate the expression of the nucleic acid molecule, one could insert a sense sequence and the appropriate promoter into the vector. Promoters can be inducible or constitutive, environmentally- or developmentally-regulated, or cell- or tissue-specific. Transcription is enhanced with promoters known in the art for expression. The CMV and SV40 promoters are commonly used to express desired polypeptide in mammalian cells. Other promoters known in the art may also be used (many suitable promoters and vectors are described in the applications and patents referenced in this application).

If one were to downregulate the expression of the nucleic acid molecule, one could insert the antisense sequence and the appropriate promoter into the vehicle. The nucleic acid molecule may be either isolated from a native source (in sense or antisense orientations), synthesized, or it may be a mutated native or synthetic sequence or a combination of these.

Examples of regulatory elements include a transcriptional promoter and enhancer or RNA polymerase binding sequence, a ribosomal binding sequence, including a translation initiation signal. Additionally, depending on the vector employed, other genetic elements, such as selectable markers, may be incorporated into the recombinant molecule. Other regulatory regions that may be used include an enhancer domain and a termination region. The regulatory elements may be from animal, plant, yeast, bacterial, fungal, viral, avian, insect or other sources, including synthetically produced elements and mutated elements.

In addition to using the expression vectors described above, the polypeptide may be expressed by inserting a recombinant nucleic acid molecule in a known expression system derived from bacteria, viruses, yeast, mammals, insects, fungi or birds. The recombinant molecule may be introduced into the cells by techniques such as Agrobacterium tumefaciens-mediated transformation, particle-bombardment-mediated transformation, direct uptake, microinjection, coprecipitation, transfection and electroporation depending on the cell type. Retroviral vectors, adenoviral vectors, Adeno Associated Virus (AAV) vectors, DNA virus vectors and liposomes may be used. Suitable constructs are inserted in an expression vector, which may also include markers for selection of transformed cells. The construct may be inserted at a site created by restriction enzymes.

In one embodiment of the invention, a cell is transfected with a nucleic acid molecule of the invention inserted in an expression vector to produce cells expressing a polypeptide encoded by the nucleic acid molecule.

Another embodiment of the invention relates to a method of transfecting a cell with a nucleic acid molecule of the invention, inserted in an expression vector to produce a cell expressing the GRF4 polypeptide or other polypeptide of the invention. The invention also relates to a method of expressing the polypeptides of the invention in a cell. A preferred process would include culturing a cell including a recombinant DNA vector including a nucleic acid molecule encoding GRF4 (or another nucleic acid molecule of the invention) in a culture medium so that the polypeptide is expressed. The process preferably further includes recovering the polypeptide from the cells or culture medium.

Probes

The invention also includes oligonucleotide probes made from the cloned GRF4 nucleic acid molecules described in this application or other nucleic acid molecules of the invention, such as Clone 7.7 (see materials and methods section). The probes may be 15 to 30 nucleotides in length and are preferably at least 30 or more nucleotides. A preferred probe is at least 15 nucleotides of GRF4 in [SEQ ID NO: 1] or the Clone 7.7 sequence. The invention also includes at least 30 consecutive nucleotides of [SEQ ID NO: 1] or the Clone 7.7 sequence. The probes are useful to identify nucleic acids encoding GRF4 peptides, polypeptides and polypeptides other than those described in the application, as well as peptides, polypeptides and polypeptides functionally equivalent to GRF4. The oligonucleotide probes are capable of hybridizing to the sequence shown in [SEQ ID NO: 1] under stringent hybridization conditions. A nucleic acid molecule encoding a polypeptide of the invention may be isolated from other organisms by screening a library under moderate to high stringency hybridisation conditions with a labeled probe. The activity of the polypeptide encoded by the nucleic acid molecule is assessed by cloning and expression of the DNA. After the expression product is isolated the polypeptide is assayed for GRF4 activity as described in this application.

Functionally equivalent GRF4 nucleic acid molecules from other cells, or equivalent GRF4 -encoding cDNAs or synthetic DNAs, can also be isolated by amplification using Polymerase Chain Reaction (PCR) methods. Oligonucleotide primers, such as degenerate primers, based on [SEQ ID NO: 2] can be prepared and used with PCR and reverse transcriptase (E. S. Kawasaki (1990), In innis et al., Eds., PCR Protocols, Academic Press, San Diego, Chapter 3, p. 21) to amplify functional equivalent DNAs from genomic or cDNA libraries of other organisms. The oligonucleotides can also be used as probes to screen cDNA libraries.

Functionally equivalent peptides, polypeptides and proteins

The present invention includes not only the polypeptides encoded by the sequences of the invention, but also functionally equivalent peptides, polypeptides and proteins that exhibit the same or similar GRF4 polypeptide activity. A polypeptide is considered to possess a function substantially equivalent to that of the GRF4 polypeptide if it has GRF4 activity. Functionally equivalent peptides, polypeptides and proteins include peptides, polypeptides and proteins that have the same or similar protein activity as GRF4 when assayed, i.e. they are able to activate Ras. A polypeptide has GRF4 activity if it is active in catalyzing guanine-nucleotide exchange on small GTPase Ras using the in-vitro GEF assay. (Where only one or two of the terms peptides, polypeptides and proteins is referred to, it will be clear to one skilled in the art whether the other types of amino acid sequences also would be useful.)

These peptides, polypeptides and proteins can contain a region or moiety exhibiting sequence identity to a corresponding region or moiety of the GRF4 polypeptide described in the application, but this is not required as long as they exhibit the same or similar GRF4 activity.

Identity refers to the similarity of two polypeptides or proteins that are aligned so that the highest order match is obtained. Identity is calculated according to methods known in the art, such as the Clustal W program. For example, if a polypeptide (called “Sequence A”) has 90% identity to a portion of the polypeptide in [SEQ ID NO: 2], then Sequence A will be identical to the referenced portion of the polypeptide in [SEQ ID NO: 2], except that Sequence A may include up to 10 point mutations, such as substitutions with other amino acids, per each 100 amino acids of the referenced portion of the polypeptide in sequence (a) in [SEQ ID NO: 2]. Peptides, polypeptides and proteins functional equivalent to the GRF4 polypeptides can occur in a variety of forms as described below.

Peptides, polypeptides and proteins biologically functional equivalent to GRF4 polypeptide include amino acid sequences containing amino acid changes in the GRF4 sequence. The functional equivalent peptides, polypeptides and proteins have at least about 40% sequence identity, preferably at least about 60%, at least about 75%, at least about 80%, at least about 90% or at least about 95% sequence identity, to the naturally GRF4 polypeptide or a corresponding region. More preferably, the functional equivalent peptides, polypeptides and proteins have at least about 97%, 98%, 99%, 99.5%, 99.9% or 99.95% sequence identity to the naturally occurring GRF4 polypeptide or a region of the sequence (such as one of the conserved domains of the polypeptide), without being identical to the sequence in [SEQ ID NO: 2]. “Sequence identity” is preferably determined by the Clustal W program. Most preferably, 1, 2, 3, 4, 5, 5-10, 10-15, 15-25 or 25-50 amino acids are modified. The sequences preferably include all the GRF4 domains and motifs described above. One or more domain or motif may be omitted to obtain desired activity. The CDC25 domain is preferably conserved in the polypeptide in order to preserve GRF4 activity. Structurally conserved regions 1, 2 and 3 (FIG. 4A) are critical for CDC25 structure and activity. Preferably, conserved amino acids in these regions would not be altered. One would be able to make more changes to the amino acid sequences in regions outside of the conserved regions of GRF4. The CDC25 region of the polypeptide includes amino acid no. 712 to amino acid no. 1006 (preferred percentages of identity are below). The invention includes polypeptides about: <350 amino acids (preferably about 294 amino acids), <500 amino acids, <750 amino acids, <1000 amino acids, <1250 amino acids, <1500 amino acids or <2000 amino acids which have sequence identity to the CDC25 region and have CDC25 activity or CDC25-like activity (preferably Ras activation).

The invention includes peptides, proteins or proteins which retain the same or similar activity as all or part of GRF4. Such peptides preferably consist of at least 5 amino acids. In preferred embodiments, they may consist of 6 to 10, 11 to 15, 16 to 25 or 26 to 50, 50 to 150, 150 to 250, 250 to 500, 500 to 750 or 750 to 1250 amino acids of GRF4. Fragments of the GRF4 polypeptide can be created by deleting one or more amino acids from the N-terminus, C-terminus or an internal region of the polypeptide (or combinations of these), so long as the fragments retain the same or similar GRF4 activity as all or part of the GRF4 polypeptide disclosed in the application. These fragments can be generated by restriction nuclease treatment of an encoding nucleic acid molecule. Alternatively, the fragments may be natural mutants of the GRF4. Fragments of the polypeptide may be used in an assay to identify compounds that bind the polypeptide. Methods known in the art may be used to identify agonists and antagonists of the fragments.

Variants of the GRF4 polypeptide may also be created by splicing. A combination of techniques known in the art may be used to substitute, delete or add amino acids. For example, a hydrophobic residue such as methionine can be substituted for another hydrophobic residue such as alanine. An alanine residue may be substituted with a more hydrophobic residue such as leucine, valine or isoleucine. An aromatic residue such as phenylalanine may be substituted for tyrosine. An acidic, negatively charged amino acid such as aspartic acid may be substituted for glutamic acid. A positively charged amino acid such as lysine may be substituted for another positively charged amino acid such as arginine. Modifications of the polypeptides of the invention may also be made by treating a polypeptide of the invention with an agent that chemically alters a side group, for example, by converting a hydrogen group to another group such as a hydroxy or amino group.

Peptides having one or more D-amino acids are contemplated within the invention. Also contemplated are peptides where one or more amino acids are acetylated at the N-terminus. Those skilled in the art recognize that a variety of techniques are available for constructing peptide mimetics (i.e. a modified peptide or polypeptide or protein) with the same or similar desired biological activity as the corresponding polypeptide of the invention but with more favorable activity than the polypeptide with respect to characteristics such as solubility, stability, andlor susceptibility to hydrolysis and proteolysis. See for example, Morgan and Gainor, Ann. Rep. Med. Chem., 24:243-252 (1989).

The invention also includes hybrid nucleic acid molecules and peptides, for example where a nucleic acid molecule from the nucleic acid molecule of the invention is combined with another nucleic acid molecule to produce a nucleic acid molecule which expresses a fusion peptide. A preferred fusion polypeptide includes all or part of the active as CDC25 Domain of GRF4. One or more of the other domains of GRF4 described in this application could also be used to make fusion polypeptides. For example, a nucleotide domain from a molecule of interest may be ligated to all or part of a nucleic acid molecule encoding GRF4 polypeptide (or a molecule having sequence identity) described in this application. Fusion nucleic acid molecules and peptides can also be chemically synthesized or produced using other known techniques. The invention includes a nucleic acid molecule encoding a fusion polypeptide or a recombinant vector including the sequence of [SEQ ID NO: 1] or [SEQ ID NO: 3]. The invention also includes a fusion polypeptide including the sequence of [SEQ ID NO: 2] or a polypeptide encoded by [SEQ ID NO: 3].

The variants preferably retain the same or similar GRF4 activity as the naturally occurring GRF4. The, GRF4 activity of such variants can be assayed by techniques described in this application and known in the art.

Variants produced by combinations of the techniques described above but which retain the same or similar GRF4 activity as naturally occurring GRF4 are also included in the invention (for example, combinations of amino acid additions, deletions, and substitutions).

Fragments and variants of GRF4 encompassed by the present invention preferably have at least about 40% sequence identity, preferably at least about 60%, 75%, 80%, 90% or 95% sequence identity, to the naturally occurring polypeptide, or corresponding region or moiety. Most preferably, the fragments have at least about 97%, 98% or 99%, 99.5%, 99.9% or 99.99% sequence identity to the naturally occurring GRF4 polypeptide, or corresponding region. Sequence identity is preferably measured with the Clustal W.

The invention also includes fragments of the polypeptides of the invention which do not retain the same or similar activity as the complete polypeptides but which can be used as a research tool to characterize the polypeptides of the invention.

Enhancement of GRF4 polypeptide activity

The activity of the GRF4 polypeptide is increased by carrying out selective site-directed mutagenesis. Using protein modeling and other prediction methods, we characterize the binding domain and other critical amino acid residues in the polypeptide that are candidates for mutation, insertion andlor deletion. A DNA plasmid or expression vector containing the GRF4 nucleic acid molecule or a nucleic acid molecule having sequence identity is preferably used for these studies using the U.S.E. (Unique site elimination) mutagenesis kit from Pharmacia Biotech or other mutagenesis kits that are commercially available, or using PCR. Once the mutation is created and confirmed by DNA sequence analysis, the mutant polypeptide is expressed using an expression system and its activity is monitored. This approach is useful not only to enhance activity, but also to engineer some functional domains for other properties useful in the purification or application of the polypeptides or the addition of other biological functions. It is also possible to synthesize a DNA fragment based on the sequence of the polypeptides that encodes smaller polypeptides that retain activity and are easier to express. It is also possible to modify the expression of the cDNA so that it is induced under desired environmental conditions or in response to different chemical inducers or hormones. It is also possible to modify the DNA sequence so that the polypeptide is targeted to a different location. All these modifications of the DNA sequences presented in this application and the polypeptides produced by the modified sequences are encompassed by the present invention.

Pharmaceutical compositions

The GRF4 nucleic acid molecule or its polypeptide and functional equivalent nucleic acid molecules or polypeptides are also useful when combined with a carrier in a pharmaceutical composition. Suitable examples of vectors for GRF4 are described above. The compositions are useful when administered in methods of medical treatment of a disease, disorder or abnormal physical state characterized by insufficient GRF4 expression or inadequate levels or activity of GRF4 polypeptide by increasing expression, concentration or activity. The invention also includes methods of medical treatment of a disease, disorder or abnormal physical state characterized by excessive GRF4 expression or levels or activity of GRF4 polypeptide, for example by administering a pharmaceutical composition including a carrier and a vector that expresses GRF4 antisense DNA. Cancer is one example of a disease which can be treated by antagonizing GRF4. An agent that upregulates GRF4 gene expression or GRF4 polypeptide activity may be combined with a carrier to form a pharmaceutical composition. An agent that downregulates GRF4 expression or GRF4 polypeptide activity may be combined with a carrier to form a pharmaceutical composition.

The pharmaceutical compositions of this invention are used to treat patients having degenerative diseases, disorders or abnormal physical states such as cancer. For example, cancer can be treated by antagonizing GRF4, by blocking CDC25 activity. The following U.S. patents deal with the use of compounds that modulate Ras in order to treat diseases, disorders or abnormal physical states: 5856439, 5852034, 5843941, 5840683, 5807853, 5801175, 5789438, 5776902, 5756528, 5712280, 5710171, 5672611, 5668171, 5663193, 5661128, 5627202, 5624936, 5585359, 5582995, 5576293, 5571835, 5567729, 5536750, 5523456, 5491164, 5480893, 5468733, 5238922, 5185248, 5523456, 5491164, 5480893, 5468733, 5238922 and 5185248 which are incorporated by reference in their entirety. The following WIPO PCT patent applications disclose the use of compounds that modulate Ras in order to treat diseases: WO9857990, WO9805786, WO9828980, WO9815556, WO9857970, WO9857964, WO9857963, WO9857949, WO9857948, WO9857947, WO9857946, WO9849194, WO9811106, WO9811098, WO9811097, WO9809641, WO9804545, WO9721820, WO9857950 and WO9737678 which are incorporated by reference in their entirety. Many of these patents and applications describe inhibition of Ras to treat excessive cell proliferation and cancer. The patents and applications disclose research techniques to identify compounds which inhibit Ras or compounds that regulate Ras.

The pharmaceutical compositions can be administered to humans or animals by methods such as tablets, aerosol administration, intratracheal instillation and intravenous injection in methods of medical treatment involving upregulating or downregulating GRF4 gene or polypeptide to upregulate or downregulate Ras activity. Dosages to be administered depend on patient needs, on the desired effect and on the chosen route of administration.

Nucleic acid molecules and polypeptides may be introduced into cells using in vivo delivery vehicles such as liposomes. They may also be introduced into these cells using physical techniques such as microinjection and electroporation or chemical methods such as coprecipitation or using liposomes.

The pharmaceutical compositions can be prepared by known methods for the preparation of pharmaceutically acceptable compositions which can be administered to patients, and such that an effective quantity of the nucleic acid molecule or polypeptide is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA).

On this basis, the pharmaceutical compositions could include an active compound or substance, such as a GRF4 nucleic acid molecule or polypeptide, in association with one or more pharmaceutically acceptable vehicles or diluents, and contained in buffered solutions with a suitable pH and isoosmotic with the physiological fluids. The methods of combining the active molecules with the vehicles or combining them with diluents is well known to those skilled in the art. The composition could include a targeting agent for the transport of the active compound to specified sites within tissue.

Administration of GRF4 nucleic acid molecule

Since persons suffering from disease, disorder or abnormal physical state can be treated by either up or down regulation of GRF4, gene therapy to increase or reduce GRF4 expression is useful to modify the development/progression of disease. For example, to treat cancer, GRF4 could be modulated to suppress Ras activity (inhibiting GRF4 prevents Ras activation).

The invention also includes methods and compositions for providing gene therapy for treatment of diseases, disorders or abnormal physical states characterized by insufficient GRF4 expression or inadequate levels or activity of GRF4 polypeptide (see the discussion of phamaceutical compositions, above) involving administration of a pharmaceutical composition of the invention. The invention also includes methods and compositions for providing gene therapy for treatment of diseases, disorders or abnormal physical states characterized by excessive GRF4 expression or levels of activity of GRF4 polypeptide involving administration of a pharmaceutical composition.

The invention includes methods and compositions for providing a nucleic acid molecule encoding GRF4 or functional equivalent nucleic acid molecule to the cells of an individual such that expression of GRF4 in the cells provides the biological activity or phenotype of GRF4 polypeptide to those cells (preferably Ras activation). Sufficient amounts of the nucleic acid molecule are administered and expressed at sufficient levels to provide the biological activity or phenotype of GRF4 polypeptide to the cells. For example, the method can preferably involve a method of delivering a nucleic acid molecule encoding GRF4 to the cells of an individual having a disease, disorder or abnormal physical state, comprising administering to the individual a vector comprising DNA encoding GRF4. The method may also relate to a method for providing an individual having a disease, disorder or abnormal physical state with biologically active GRF4 polypeptide by administering DNA encoding GRF4. The method may be performed ex vivo or in vivo. Methods and compositions for administering GRF4 (including in gene therapy) are explained, for example, in U.S. Pat. Nos. 5,672,344, 5,645,829, 5,741,486, 5,656,465, 5,547,932, 5,529,774, 5,436,146, 5,399,346 and 5,670,488, 5,240,846 which are incorporated by reference in their entirety.

The method also relates to a method for producing a stock of recombinant virus by producing virus suitable for gene therapy comprising DNA encoding GRF4. This method preferably involves transfecting cells permissive for virus replication (the virus containing the nucleic acid molecule) and collecting the virus produced.

The invention also includes methods and compositions for providing a nucleic acid molecule encoding an antisense sequence to GRF4 or a Nedd4 nucleic acid molecule sequence to the cells of an individual such that expression of the sequence prevents GRF4 ybiological activity or phenotype or reduces GRF4. The methods and compositions can be used in vivo or in vitro. Sufficient amounts of the nucleic acid molecule are administered and expressed at sufficient levels to reduce the biological activity or phenotype of GRF4 polypeptide in the cells. Similar methods as described in the preceding paragraph may be used with appropriate modifications.

The methods and compositions can be used in vivo or in vitro. The invention also includes compositions (preferably pharmaceutical compositions for gene therapy). The compositions include a vector containing GRF4. Nedd4 or a functional equivalent molecule or antisense DNA. The carrier may be a pharmaceutical carrier or a host cell transformant including the vector. Vectors known in the art include adenovirus, adeno associated virus (AAV), herpes virus vectors, such as vaccinia virus vectors, and plasmids. The invention also includes packaging cell lines that produce the vector. Methods of producing the vector and methods of gene therapy using the vector are also included with the invention.

The invention also includes a transformed cell, such as a brain cell or a lung cell containing the vector and recombinant GRF4 nucleic acid molecule antisense sequence, Nedd4 or a functionally equivalent molecule.

Heterologous expression of GRF4

Expression vectors are useful to provide high levels of polypeptide expression. Cell cultures transformed with the nucleic acid molecules of the invention are useful as research tools particularly for studies of GRF4 interactions with Ras. Novel pathways to activate Ras are identified. Cell cultures are used in overexpression and research according to numerous techniques known in the art. For example, a cell line (either an immortalized cell culture or a primary cell culture) may be transfected with a vector containing a GRF4 nucleic acid molecule (or molecule having sequence identity) to measure levels of expression of the nucleic acid molecule and the activity of the nucleic acid molecule and polypeptide. A polypeptide of the invention may be used in an assay to identify compounds that bind the polypeptide. Methods known in the art may be used to identify agonists and antagonists of the polypeptides. One may obtain cells that do not express GRF4 endogenously and use them in experiments to assess ectopoic GRF4 nucleic acid molecule expression. Experimental groups of cells may be transfected with vectors containing different types of GRF4 nucleic acid molecules (or nucleic acid molecules having sequence identity to GRF4 or fragments of GRF4 nucleic acid molecule) to assess the levels of polypeptide produced, its functionality and the phenotype of the cells produced. Other expression systems can also be utilized to overexpress the GRF4 in recombinant systems. The polypeptides are also useful for in vitro analysis of GRF4 activity. For example, the polypeptide produced can be used for microscopy or X-ray crystallography studies, and the tertiary structure of individual domains may be analyzed by NMR spectroscopy.

Experiments may be performed with cell cultures or in vivo to identify polypeptides that bind to different domains of GRF4. One could also target cNMP to block upstream activators or inhibitors. Nedd4 binding to GRF4 can be studied. For example, Nedd4 binding could be blocked to study the effects on GRF4 stability. Another example is blocking the PDZ domain to prevent membrane localization of GRF4. Similar approaches could be taken to study other polypeptide domains or motifs.

Preparation of antibodies

The GRF4 polypeptide is also useful as an antigen for the preparation of antibodies that can be used to purify or detect other GRF4like polypeptides. To recognize the polypeptide: preferably target to the C-terminus. To block activity: preferably target to the CDC25 domain, to block aCMP/cGMP-dependent activity, preferably target the CNMP-BD. To block membrane targeting: preferably target to the PDZ domain.

We have already generated polyclonal antibodies against the C-terminal 150 amino acids of GRF4 which is a unique region. Monoclonal and polyclonal antibodies are prepared according to the description in this application and techniques known in the art. For examples of methods of the preparation and uses of monoclonal antibodies, see U.S. Pat. Nos. 5,688,681, 5,688,657, 5,683,693, 5,667,781, 5,665,356, 5,591,628, 5,510,241, 5,503,987, 5,501,988, 5,500,345 and 5,496,705 which are incorporated by reference in their entirety. Examples of the preparation and uses of polyclonal antibodies are disclosed in U.S. Pat. Nos. 5,512,282, 4,828,985, 5,225,331 and 5,124,147 which are incorporated by reference in their entirety. Antibodies recognizing GRF4 can be employed to screen organisms or tissues containing GRF4 polypeptide or GRF4-like polypeptides. The antibodies are also valuable for immuno-purification of GRF4 or GRF4-like polypeptides from crude extracts.

An antibody (preferably the antibody described above) may be used to detect GRF4 or a similar polypeptide, for example, by contacting a biological sample with the antibody under conditions allowing the formation of an immunological complex between the antibody and a polypeptide recognized by the antibody and detecting the presence or absence of the immunological complex whereby the presence of GRF4 or a similar polypeptide is detected in the sample. The invention also includes compositions preferably including the antibody, a medium suitable for the formation of an immunological complex between the antibody and a polypeptide recognized by the antibody and a reagent capable of detecting the immunolgical complex to ascertain the presence of GRF4 or a similar polypeptide. The invention also includes a kit for the in vitro detection of the presence or absence of GRF4 or a similar polypeptide in a biological sample, wherein the kit preferably includes an antibody, a medium suitable for the formation of an immunological complex between the antibody and a polypeptide recognized by the antibody and a reagent capable of detecting the immunological complex to ascertain the presence of GRF4 or a similar polypeptide in a biological sample. Further background on the use of antibodies is provided, for example in U.S. Pat. Nos. 5,695,931 and 5,837,472 which are Incorporated by reference in their entirety.

Diagnostic test

In many cancers, Ras is aberrantly expressed or is mutated. It is likely that in some cancers, GRF4 is mutated as well, so GRF4 is useful as a screening tool for the detection of cancer or to monitor its progression. For example, GRF4 may be sequenced to determine if a cancer-causing mutation is present. Levels of GRF4 may also be measured to determine whether GRF4 is upregulated. A cancer causing mutation or upregulated levels are indicative of cancer.

Screening for agonists and antagonists of GRF4 nucleic acid molecule and enhancers and inhibitors of GRF4 polypeptide

Inhibitors are preferably directed towards specific domains of GRF4 to block Ras activation. To achieve specificity, inhibitors should target the unique sequences of GRF4. For example, (i) they should block the cNMP-BD of GRF4 but not the cAMP binding site of protein kinase A (PKA) or protein kinase G (PKG), (ii) they could interfere with targeting of the PDZ domain to the plasma membrane, where Ras (the GRF4 substrate) is located or (iii) they could target the unique insert sequence within the CDC25 (catalytic) domain of GRF4. A similar approach can be used to search for compounds that may enhance Ras activation by GRF.

A method of identifying a compound which modulates the interaction of GRF4 with Ras, can include:

a) contacting (i) GRF4, a Ras-binding fragment of GRF4 (eg, the CDC25-BD, or part of the domain, such as a part including the unique 40 amino acid insert) or a derivative of either of the foregoing with (ii) Ras, a GRF4-binding fragment of Ras or a derivative of either of the foregoing in the presence of the compound; and b) determining whether the interaction between (i) and (ii) is modulated, thereby indicating that the compound modulates the interaction of GRF4 and Ras. Similar methods may be performed using Rap1.

Modulation can include increasing or decreasing the interaction between (i) and (ii). A GRF4 or Ras inhibitor (anti-cancer or anti-proliferative compound) inhibits the interaction between (i) and (ii).

The method preferably includes identifying a compound that blocks the cNMP-BD of GRF4 but not the cAMP binding site of protein kinase A (PKA) or protein kinase G (PKG). The method may alternatively include identifying a compound that interferes with targeting of the PDZ domain to the plasma membrane, where Ras (the GRF4 substrate) is located. The method may alternatively include identifying a compound that interferes with the unique insert sequence within the CDC25 (catalytic) domain of GRF4. A similar approach can be used to search for compounds that may enhance Ras activation by GRF4. More detailed methods of screening are described below.

Methods of screening

Small molecules or peptides

By way of example, one can screen either (i) synthetic peptide library, as described by Songyang et al, Cell 72:767, 1993 and Songyang et al, Science 275:73, 1997, for the identification of sequences recognized by the SH2 or PDZ domains, respectively or (ii) Phagedispiayed Random library screen, as described in Sparks et al., J. Biol. Chem. 269:23853, 1994 and Cheadle et al, J. Biol. Chem. 269:24034, 1994, used for the identification of sequences which bind to the src-SH3 domain. One could also screen small non-peptide organic molecules.

Thus, the invention includes a method of identifying a compound which modulates the interaction of GRF4 with Ras, including contacting the compound with a domain of GRF4 (such as cNMP-BD, PDZ or CDC25 domain), or a fragment or derivative thereof and determining the ability of the compound to bind to the GRF4, fragment or derivative, thereby indicating that the compound modulates the interaction of GRF4 and Ras. One may preferably target the unique sequence in the CDC25 domain of GRF4 rather than the sequences that are common to other CDC25 domains. To specifically block Ras activation by GRF4, one can target the unique sequences sof GRF4. For more general blocking of Ras (multiple pathways), portions of GRF4 similar to other CDC25 sequences (eg. similar SOS sequences (65)) may be blocked. A method may also be performed to determine whether the compound modulates the interaction of GRF4 with Ras, including: a) contacting (i) GRF4, a Ras-binding fragment of GRF4 or a derivative of either of the foregoing with (ii) Ras, a GRF4-binding fragment of Ras or a derivative of either of the foregoing in the presence of the compound; and b) determining whether the interaction between (i) and (ii) is modulated, thereby indicating that the compound modulates the interaction of GRF4 and Ras. The ability to interfere with the interaction of GRF4 with Ras indicates that the compound is useful in preventing Ras activation and cell proliferation. The compound is also useful in treatment of cancer. Similar screening methods may be performed with Rap1.

Each of the domains of GRF4 (especially the cNMP-BD, PDZ and CDC25 domain), expressed as GST fusion proteins (which we have already generated) can be incubated with such peptide libraries, to identify sequences required for binding. Again, specificity can be obtained by looking for sequences which uniquely recognize GRF4 domains (for example, peptides recognizing the cNMP-BD of GRF4 but not the cAMP-BD of PKA or the cGMP-BD of PKG or of cyclic nucleotide-gated K+ channels).

Large molecules/proteins

These molecules preferably serve as templates for generation of mimetics. To identify proteins interacting with the different domains of GRF4, several methods may be applied: (i) Expression library screen, as described in this application, (ii) yeast 2 hybrid screen (Chien et al., Proc. Nati. Acad. Sci USA, 88:9578, 1991), (iii) protein microarray chip screens. The latter will allow, once the human genome project is complete, to identify most, if not all, possible GRF4 interacting proteins in humans.

Identification of small (peptide) or large molecules which interact with GRF4 and blocks or enhances its activity and hence Ras activation

Ras plays a key role in regulation of cell proliferation, differentiation and transformation, so regulating its activity has fundamental implications for the regulation of these processes, especially in cancer development and progression.

As described above, GRF4 is useful in a pharmaceutical preparation to treat cancer and other diseases disorders and abnormal physical states. Nedd4 (preferably all or part of Nedd4, such as the GRF4 binding domain of Nedd4) is one agent which reduces GRF4 activity. cAMP and cGMP are agents which increase GRF4 activity. GRF4 is also useful as a target. Modulation of GRF4 expression is commercially useful for identification and development of drugs to inhibit and/or enhance GRF4 function directly. Such drugs would preferably be targeted to any of the following sites: CDC25 domain, PDZ domain, cNMP-BD. Chemical libraries are used to identify pharmacophores which can specifically interact with GRF4 either in an inhibitory or stimulatory mode. The GRF4 targets that would be used in drug design include the CDC25 domain, in order to inhibit its catalytic activity. For example, nucleotide analogues which stabilize the Ras-analogue complex, thus preventing replacement of the nucleotide analogue by Ras, could interfere with activation of GRF4. Similarly, other compounds directed against the binding site of Ras on GRF4 could be useful as well. The insert in the CDC25 domain in GRF4 is unique and is useful as a target. The PDZ domain is necessary for proper localization of GRF4 to the plasma membrane and is useful as a target. The cNMP binding domain is useful to disconnect GRF4 from upstream signaling. The invention also includes methods of screening a test compound to determine whether it antagonizes or agonizes GRF4 polypeptide activity. The invention also includes methods of screening a test compound to determine whether it induces or inhibits GRF4 nucleic acid molecule expression.

In a preferred embodiment, the invention includes an assay for evaluating whether test compounds are capable of acting as agonists or antagonists for GRF4, or a polypeptide having GRF4 functional activity, including culturing cells containing DNA which expresses GRF4, or a polypeptide having GRF4 activity so that the culturing is carried out in the presence of at least one compound whose ability to modulate GRF4 activity (preferably Ras activating activity or CDC25 domain activity) is sought to be determined and thereafter monitoring the cells for either an increase or decrease in the level of GRF4 or GRF4 activity. Other assays (as well as variations of the above assay) will be apparent from the description of this invention and techniques such as those disclosed in U.S. Pat. No. 5,851,788, 5,736,337and 5,767,075 which are incorporated by reference in their entirety. For example, the test compound levels may be either fixed or increase.

Localization of GRF4

i) Tissue distribution of GRF4

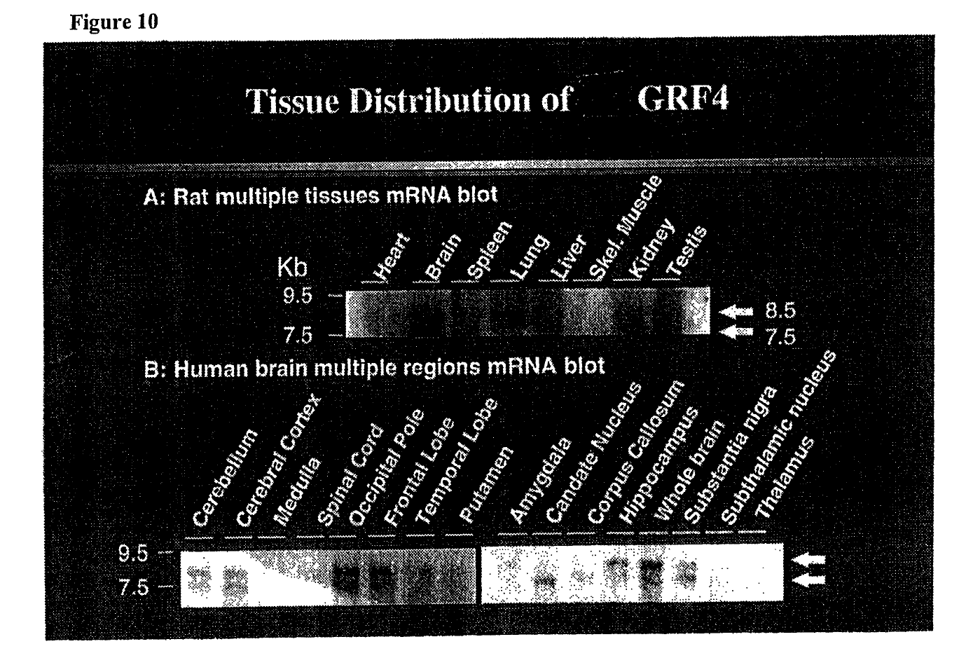

To show tissue distribution of GRF4, mouse GRF4 specific probes were used to probe a Rat multiple.tissue mRNA blot (Clonetech). Two messages, of 8.5 and 7.5 Kb, are present in rat brain; the 8.5 Kb message is also present in rat lung (FIG. 10). We determine the polypeptide's distribution in neuronal tissue. The finding of GRF4 message in rat brain is consistent with the fact that its cDNA was initially isolated from a human brain cDNA library. Using human GRF4 specific probes on the human brain multiple region mRNA blots (Clonetech), GRF4 messages (8.5 and 7.5 Kb) are found widespread (FIG. 10). The two messages may correspond to splicing variants or isoforms of GRF4. In comparison, SOS is ubiquitously expressed, whereas RasGRF1, RasGRF2 and RasGRP are expressed primarily in the brain (23,26,27). We detect GRF4 polypeptides in cell lines using known techniques.

ii) Characterization of Nedd4-GRF4 interaction

Since mouse GRF4 was isolated from the expression library screen using Nedd4-WW2 domain as a probe, further characterization of their interaction was studied.

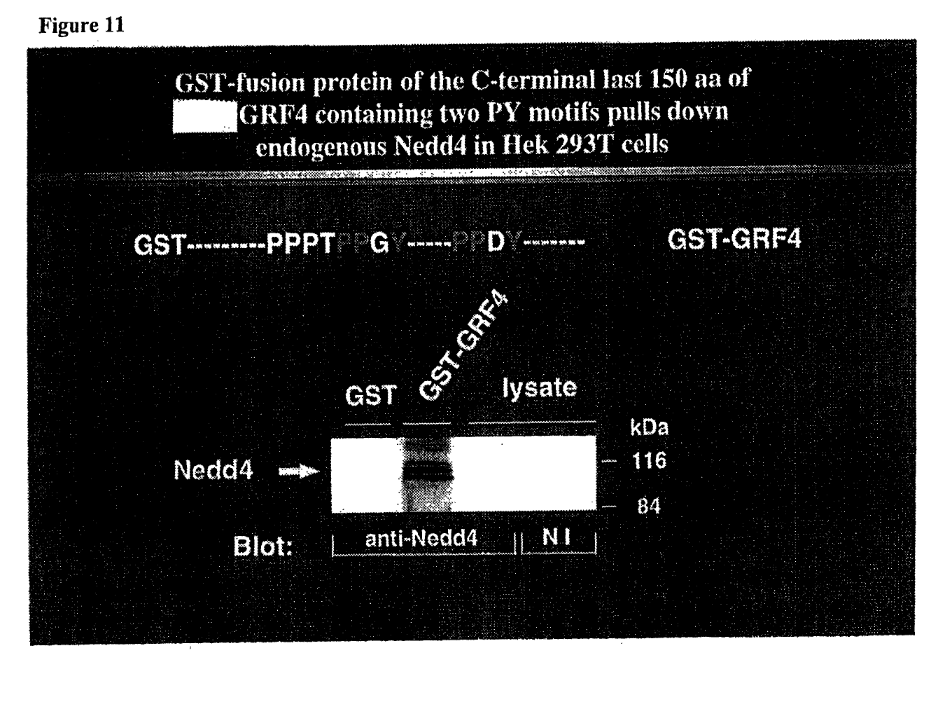

A GST-fusion protein of polypeptide corresponding to the last 150 amino acid of GRF4 (about the same length as the partial amino acid sequence isolated from the screen), containing the two PY motifs of GRF4, was generated and used in a pull-down experiment. Nedd4 is endogenously expressed in Hek 293T cells and can be detected in 293T lysates using Nedd4 antibodies (FIG. 11). When 293T lysates were incubated with agarose beads bound to GST or GST-fusion protein of the PY-containing polypeptide, Nedd4 was found to bind specifically to this polypeptide, showing that the two PY motifs of GRF4 are sufficient to interact with full-length Nedd4.

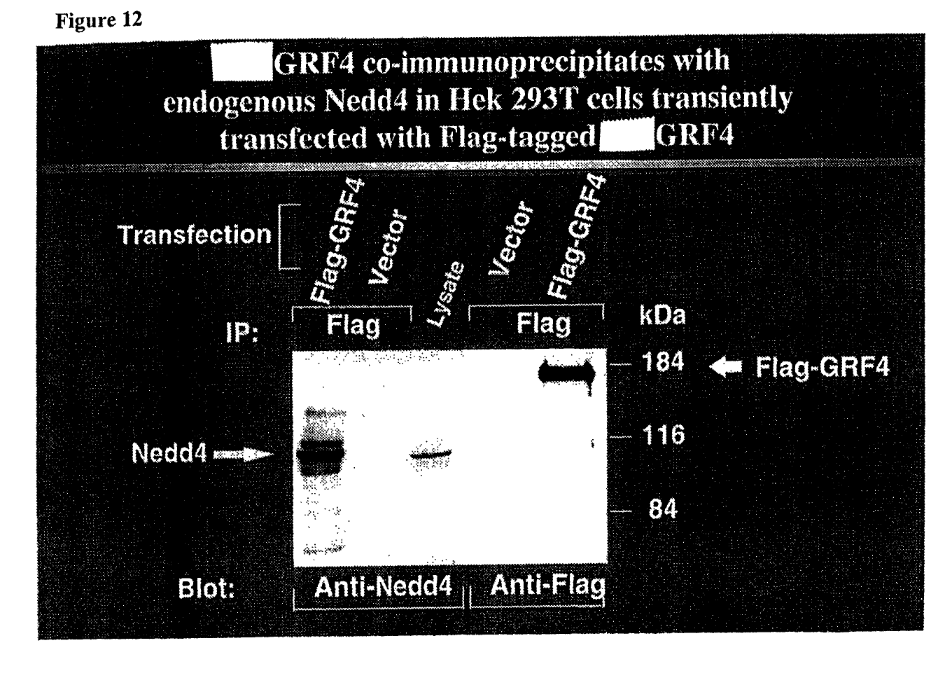

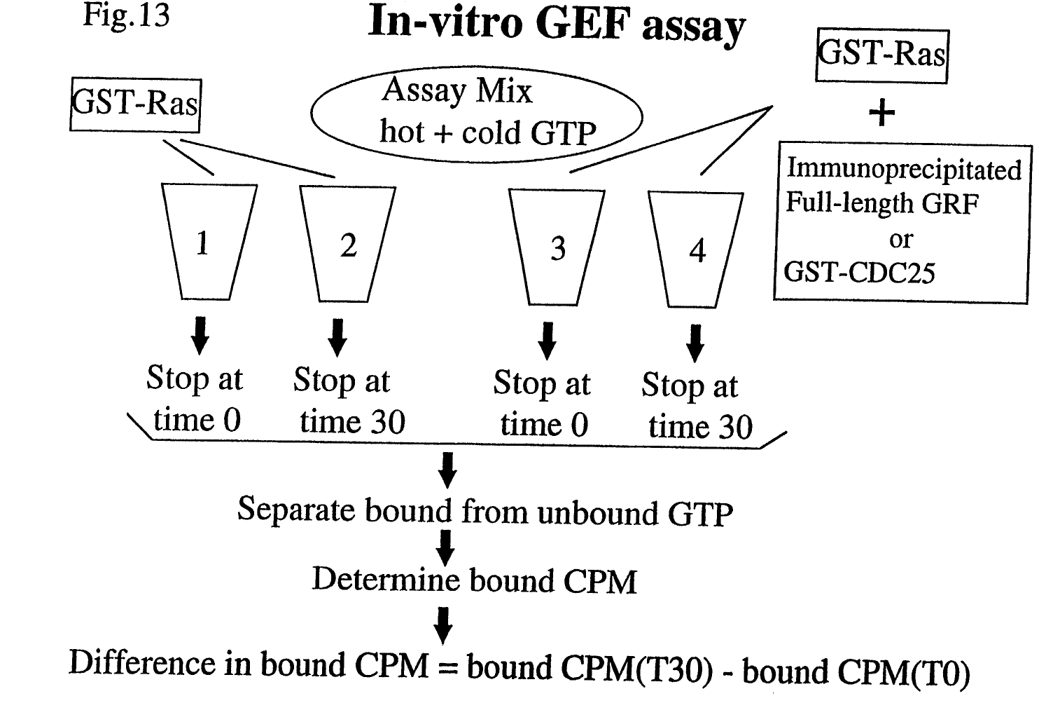

The interaction between Nedd4 and GRF4 was also demonstrated in living cells by co-immunoprecipitation. Flag-epitope tagged GRF4 was constructed in a mammalian expression vector (pCMV5). The co-immunoprecipitation experiment was performed using endogenous Nedd4 and transiently transfected Flag-tagged GRF4 in Hek 293T cells. First, Flag-tagged GRF4 was immunoprecipitated from transfected lysates using anti-Flag gel affinity (Sigma). When this immunocomplex containing GRF4 was resolved on SDS-PAGE and subsequently immunoblotted with Nedd4 antibodies, Nedd4 was detected in this immunocomplex. However, Nedd4 was not found in the immunocomplex that did not have GRF4 when lysates of cells transfected with empty vector were used (FIG. 12). Therefore, Nedd4 is co-immunoprecipitating with GRF4, showing that they interact in living cells.

GRF4 also contains PEST sequences. GRF4 is an unstable protein which is ubiquitinated by Nedd4 and targeted for degradation via the ubiquitin-dependent proteolytic pathway. We perform a ubiquitination assay to show that GRF4 is ubiquitinated protein using the protocol described in Ref. 34.

iii) In-vitro guanine nucleotide exchange activities

GRF4 has a RasGRF(GEF) activity/function. To show its GEF activity, we performed in-vitro GEF assays. The schematic outline of the in-vitro GEF assay protocol (described in Ref. 24) is given in FIG. 13. Briefly, GST-Ras was added alone (tubes 1 and 2) or along with GST-CDC25, or immunoprecipitated full-length of GRF4 (tubes 3 and 4). All tubes contained assay mixture including cold GTP and P32 alpha GTP. The exchange reactions were stopped at the indicated times. The stopped reaction mixtures were passed through nitrocellulose filters which were then washed with stop buffer to separate bound and unbound nucleotides. Filters were dried and then quantified by scintillation counting to determine bound CPM. The labeled nucleotides trapped on the washed filters were assumed to be Ras-associated. The difference in bound CPM over 30 minute period was determined for reactions where GST-Ras was added alone (it is the difference in bound CPM between tubes 1 and 2) and where GST-Ras was added with a GEF (it is the difference in bound CPM between tubes 3 and 4). The former is the basal level of GTP-binding to Ras and the later is usually increased several folds over the basal activity if the indicated GEF is active.

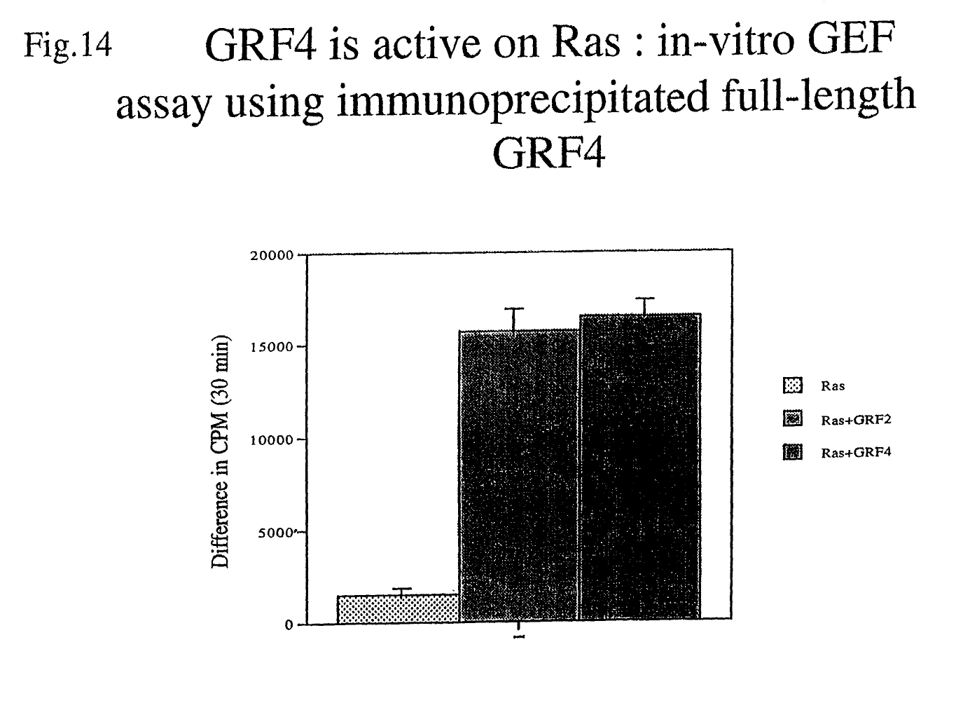

Using the GEF assay described above, the immunoprecipitated full-length GRF4 was shown to be active on Ras (FIG. 14). Additional data on the GEF assays in vitro are summarized in figure (FIG. 23e). Similar levels of GEF activity were also observed for the immunoprecipitated full-length RasGRF2 used as a positive control in this assay.

We perform in-vitro GEF assays using GST-CDC25 of GRF4 to show that this domain is sufficient for activity.

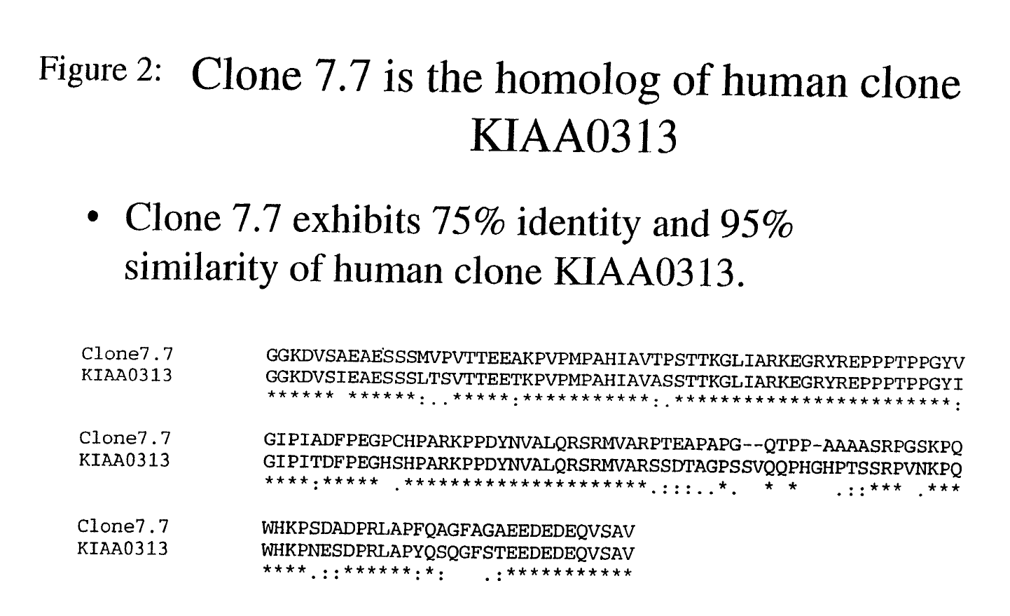

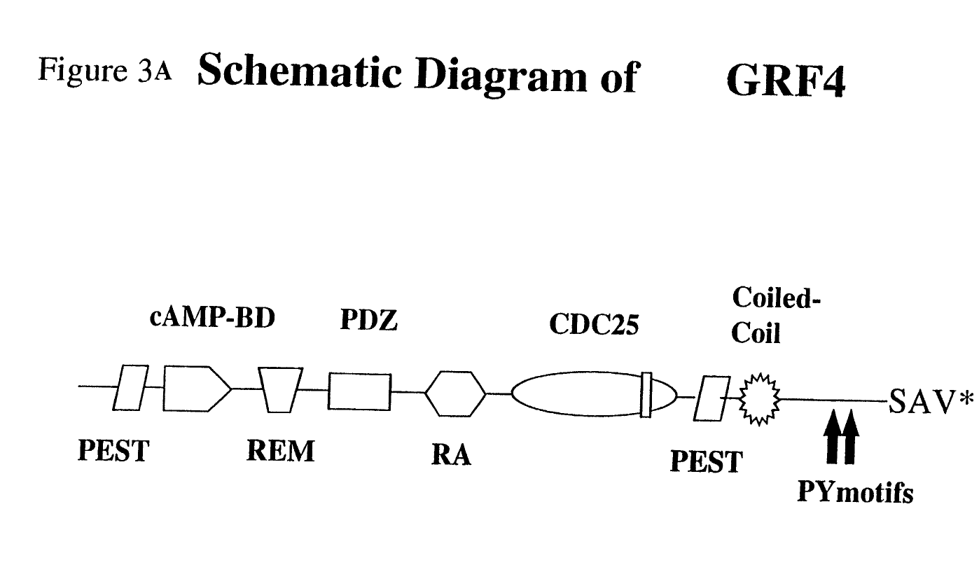

Because GRF4 contains a cNMP-BD, we tested its ability to bind cAMP directly. FIG. 2a shows that cAMP immobilized on agarose beads was able to bind the GST-cNMP-BD of GRF4 in vitro, showing a direct interaction between cAMP and GRF4-cNMP-BD. These results were substantiated by demonstrating that immobilized cAMP was able to precipitate full length GRF4 from HEK-293T cells expressing GRF4, but not from cell expressing GRF4 in which the cNMP-BD was deleted (ΔcNMP-BD) (FIG. 22b). Binding of cAMP to the GRF4-cNMP-BD was also effectively competed by cGMP (not shown), showing that cGMP can also bind to the GRF4-cNMP-BD. This is consistent with lack of conservation of the RAA and RTA sequences (described above) in the cNMP-BD, which dictates specificity towards cAMP or cGMP, respectively (Y. Su, et al., 1998).

As GRF4 contains a CDC25 domain homologous to those of GEF/GRFs for Ras and Rap families of small GTPases, we next tested the ability of GRF4 to activate Ras or Rap1 protein in cells, and whether this activation is dependent on cAMP or cGMP binding. Flag-tagged GRF4 was expressed in HEK-293T cells and its ability to activate Ras or Rap1 was analyzed by a previously described method employing activation specific probes for these GTPases: the Ras binding domain of Raf-1 that specifically binds the active, GTP-bound form of Ras, and the Rap1 binding domain of Ral-GDS which binds the GTP-bound form of Rap1 (C. Herrmann et al., 1995, J. de Rooij et al., 1998, S. J. Taylor et al., 1996). As seen in FIG. 3a, heterologous expression of GRF4 in HEK-293T cells led to a weak activation of endogenous Ras, which was greatly enhanced following stimulation of cells with the membrane permeable, nonhydrolyzable analogue of cAMP, 8-Br-cAMP. This effect was independent of PKA activity, because cAMP was still able to stimulate GRF4-mediated activation of Ras in the presence of H-89 or Rp8-Br-cAMPS, two known inhibitors of PKA (FIG. 23a). For example, in FIG. 3a, GRF4 stimulation of Ras activation was enhanced 3.9 fold following 8-Br-cAMP treatment and 3.5 fold following 8-Br-cAMP plus H-89 treatment. Similarly, 8-Br-cGMP, the membrane permeable, non-hydrolyzable analogue of cGMP, was able to greatly enhance activation of Ras by GRF4, and this effect was also independent of PKG activity, as determined by lack of effect of H-8 or RP8-Br-cGMPS, two known inhibitors of PKG (FIG. 23b). Moreover, the activation of Ras by GRF4 in cells was also seen following treatment which leads to elevation of intracellular cAMP and cGMP concentrations: namely, treating cells with forskolin plus IBMX, or YC-1 (3-(5′-Hydroxymethyl-2′-furyl)-1-benzylindazole) plus DiPyridamole (DiPy), to elevate intracellular levels of cAMP or cGMP, respectively (FIG. 23c). Thus, these results show that both cAMP and cGMP can stimulate GRF4 to activate Ras in living cells. To verify that this activation is mediated by the CDC25 domain and is dependent on the cNMP-BD of GRF4, we performed similar experiments using GRF4 lacking its CDC25 domain (ACDC25) or its cNMP-BD (ΔcNMP-BD). As seen in FIG. 23d, the 8-Br-cAMP-mediated activation of Ras via wt-GRF4 was almost abolished in the mutant GRF4 lacking its CDC25 domain or its cNMP-BD, demonstrating that the activation of Ras by GRF4 in cells requires both intact CDC25 domain and cNMP-binding domain. In vitro, we were unable to demonstrate significant activation of Ras by the isolated CDC25 domain of GRF4, but immunoprecipitated full length GRF4 (which includes also the REM domain located far upstream of the CDC25 domain, see FIG. 21a) was able to moderately enhance Ras activation (FIG. 23e), with ˜2.9 fold activation in 30 min.; this stimulation was smaller than the 4.5 fold activation conferred by GRF2, used as a control. These results show that in its native conformation in cells, GRF4 becomes fully active in response to elevated cAMP/cGMP levels, likely by direct nucleotide binding, although indirect effect can not be ruled out at present.

iv) In-vitro interaction of GRF4 with Ras

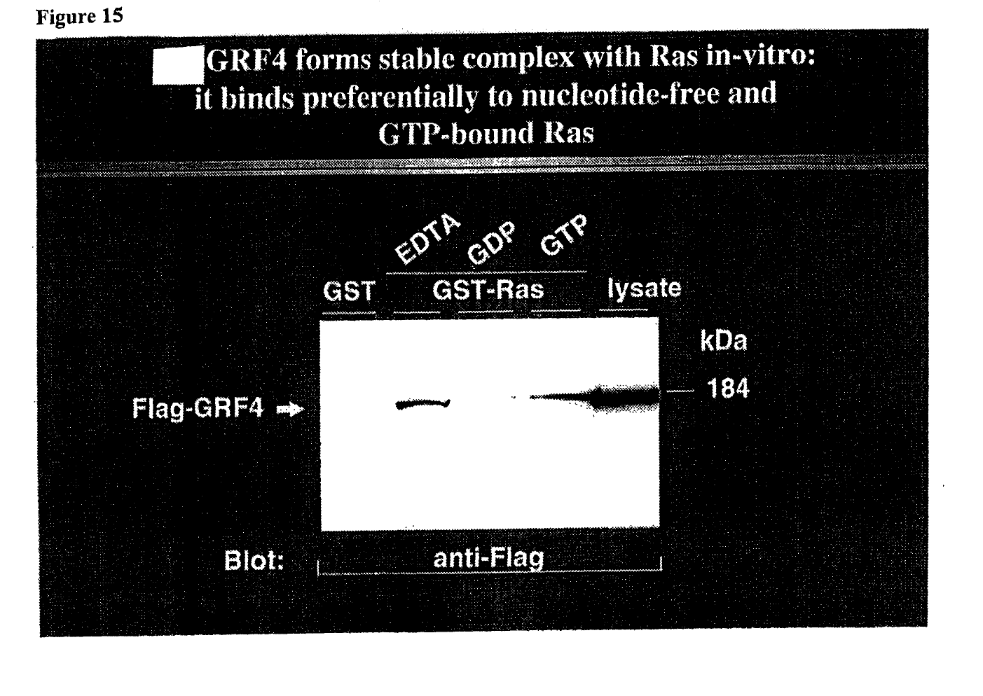

In order to show that GRF4 can form a stable complex with Ras in vitro, and which nucleotide-bound forms of Ras it binds preferentially, an in-vitro pulldown experiment was performed as follows: Lysates of 293T cells transiently transfected with Flag-tagged GRF4 were incubated with agarose beads bound to either GST alone or GST-Ras of different nucleotide-bound states. Beads were washed and resolved on SDS-PAGE and subsequently immunoblofted with anti-Flag antibodies to detect Flag-tagged GRF4. The results showed that GRF4 bound specifically to Ras as it failed to bind to GST alone. However, it bound to Ras differentially, depending on the nucleotide-bound states of Ras. GRF4 bound strongly to EDTA-treated Ras (EDTA chelates Mg2+ which is important for binding of nucleotides to Ras, thus keeps Ras in nucleotide-free form) and Ras-GTP, but bound weakly to Ras-GDP (FIG. 15). In similar experiments, RasGRF2 was shown to bind only to EDTA-treated Ras (23).

v) Activation of Ras and MAPK by cAMP and cAMP analogues:

Treatment of HEK-293T cells transfected with GRF4 with membrane permeant analogues of cAMP (8-bromo-cAMP) and cGMP (8bromo-cGMP) leads to activation of Ras and of MAPK in GRF4-expressing cells but not in untransfected cells, demonstrating that these cNMP analogues activate Ras and its downstream signaling pathway via GRF4. Moreover, a mutant GRF4 in which the cNMP-binding domain (cNMP-BD) is deleted activates Ras and MAPK constitutively, indicating that the normal function of the cNMP-BD is to suppress the activity of the CDC25 domain, an inhibition relieved by cNMP binding or by deletion of the cNMP-BD.

vi) Transformation assay

The small GTPase Ras functions as a molecular switch in cells by switching between its inactive form when it is bound to GDP and its active formn when it is bound to GTP. RasGRFs activate Ras by promoting nucleotide exchange from GDP (inactive) to GTP (active) on Ras. Active Ras activates the MAPK pathway and other signaling pathways to control normal cellular events such as cellular proliferation and differentiation. However, when Ras activity can not be deactivated as in the case of mutant oncogenic Ras, Ras becomes oncogenic and its transforming ability is the underlying mechanism of cellular transformation and is the cause of many human cancers (Ref 41-44). Several signaling proteins upstream and downstream of Ras, either controlling the activity of Ras or carrying out Ras effects, were also shown to be oncogenic.

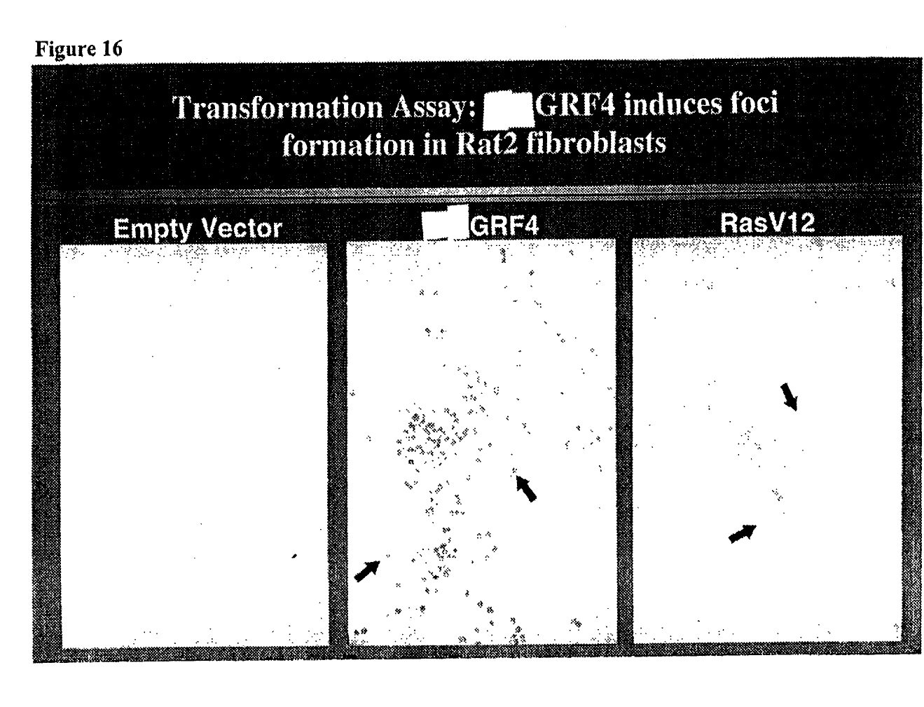

We showed that GRF4 can transform cells overexpressing this protein. Transformation assays were performed using Rat 2 fibroblasts, a suitable cell type for this assay. Rat 2 cells were transiently transfected with empty vector, GRF4 construct, or mutant RasV12 construct (a transforming form of Ras used as a positive control). After transfection, cells were cultured over a period of three weeks with routine changes of media, and were routinely examined for morphology changes under a light microscope. FIG. 16 shows the result of the assay. Rat 2 cells transfected with empty vector grew at moderate rate and maintained a monolayer state of normal saturation density, as seen with non-transfected cells. In contrast, Rat 2 cells transfected with the GRF4 construct grew faster, achieved much higher saturation density as compared to cells transfected with empty vector; more importantly, GRF4 induced foci formation in these transfected cells. A focus is the site where a single transformed cell proliferates and forms a prolific mass of transformed cells; foci formation shows a loss of cell-cell contact inhibition, a hallmark of cellular transformation. A similar phenotype was also observed with Rat 2 cells transfected with RasV12 construct. The finding that GRF4 induces foci formation in Rat 2 fibroblasts shows that GRF4 is oncogenic as well as highlights the physiological importance of this protein.

vii) PDZ domain of GRF4 interacts with its own PDZ-binding motif, SAV*

GRF4 harbours a PDZ domain and a PDZ-binding motif in context of SAV* and thus, it is involved in potential intramolecular interaction or intermolecular homotypic interaction.

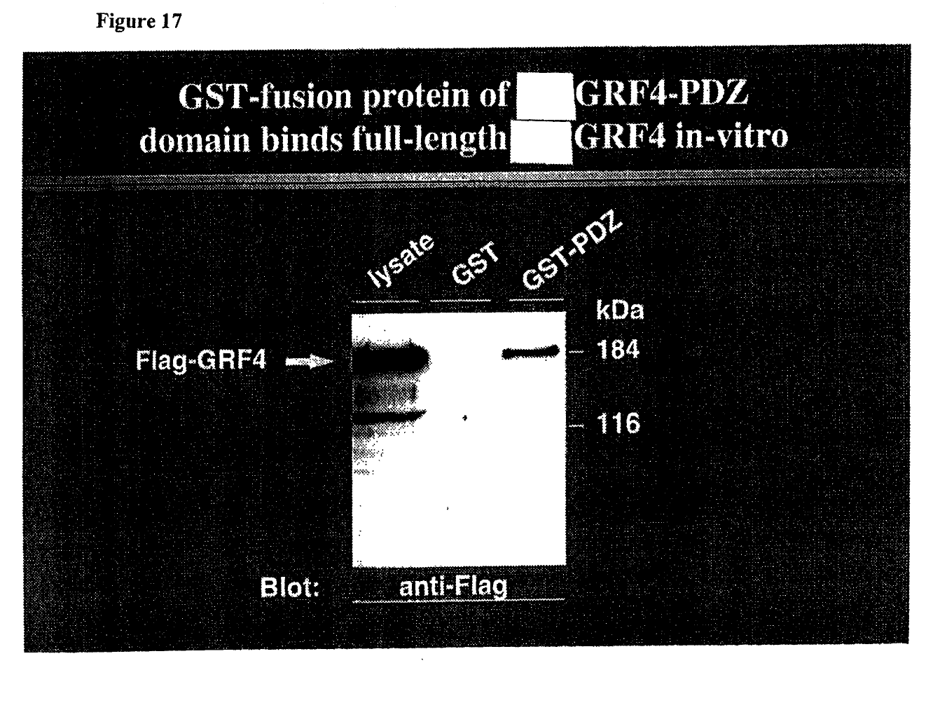

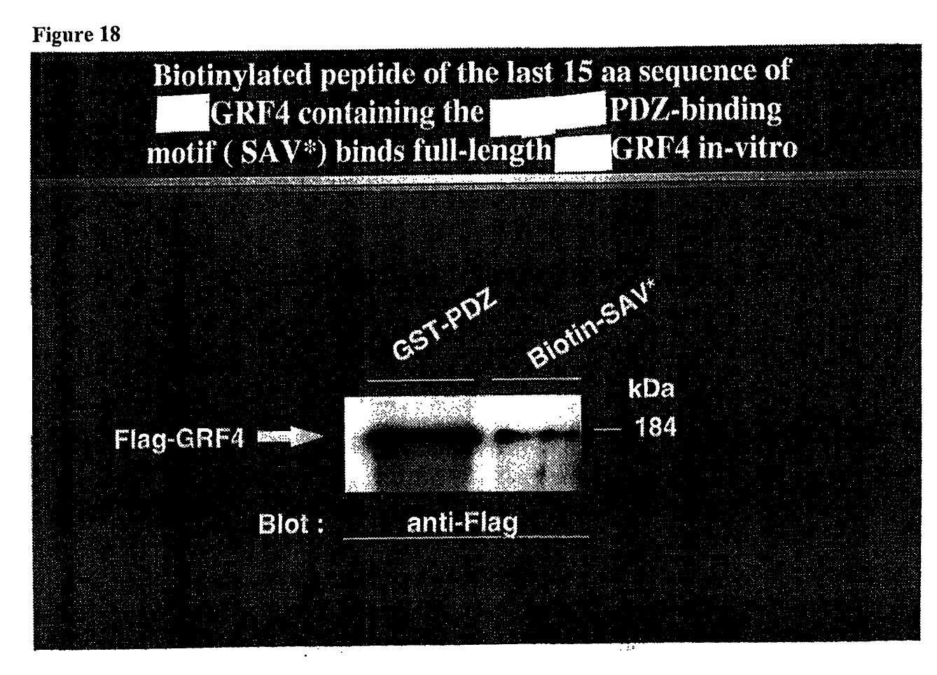

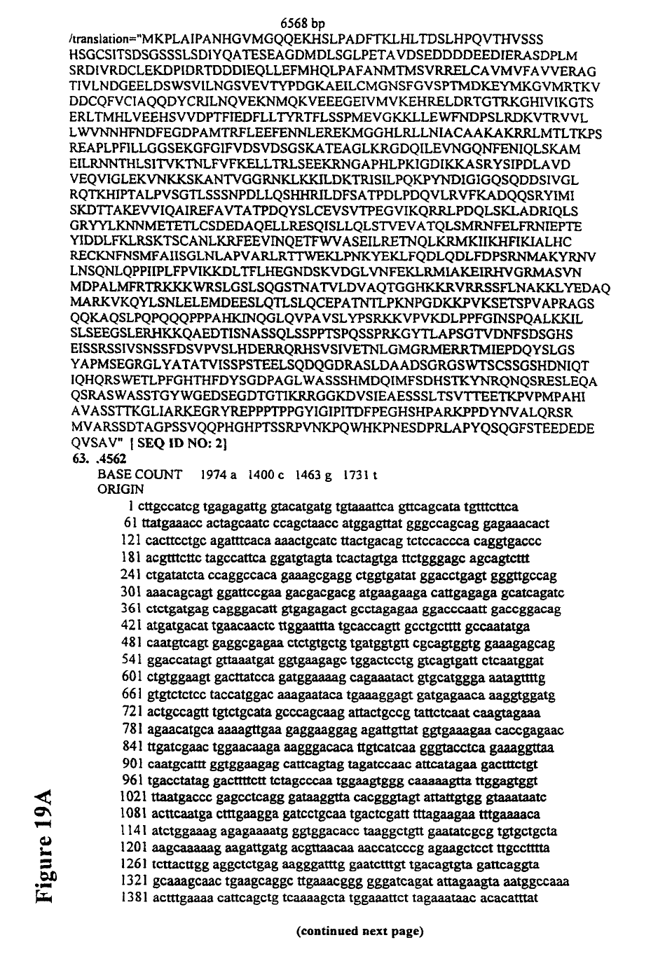

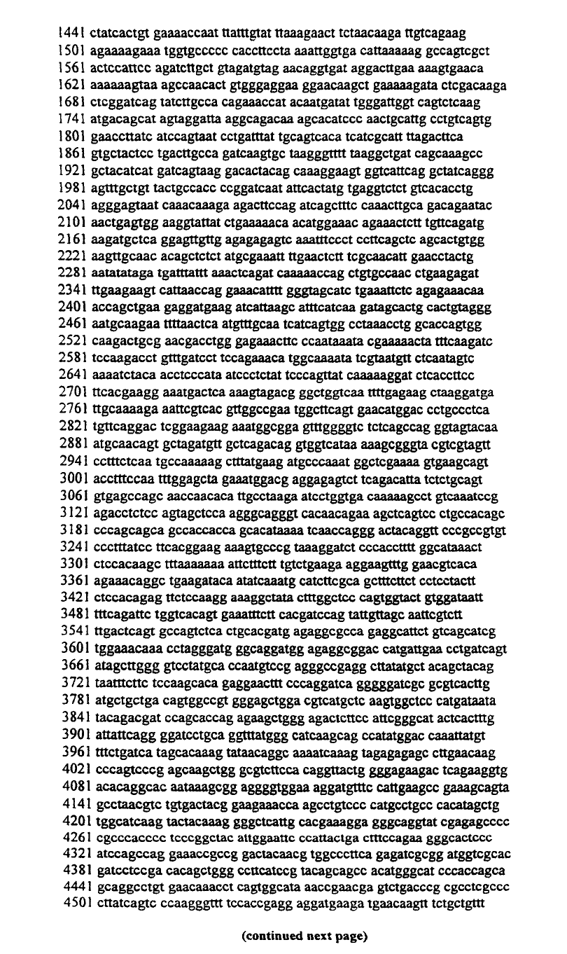

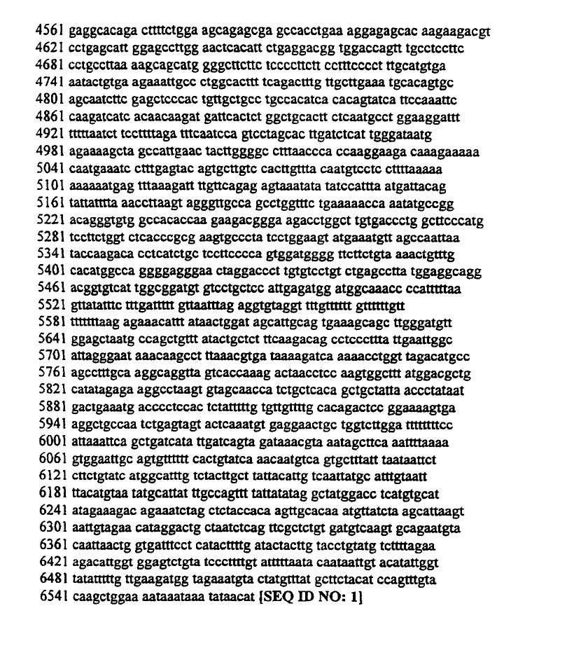

The following experiment indicates that the PDZ domain of GRF4 binds to its own SAV* motif and thus gives rise to either intramolecular interaction or intermolecular homotypic interaction. A GST-fusion protein of GRF4-PDZ domain (GST-PDZ) was generated and used in a pull-down experiment. Lysates of 293T cells transfected with Flag-tagged full-length GRF4 were incubated with agarose beads bound to GST alone or GST-PDZ. Beads were washed and resolved on SDS-PAGE and subsequently immunoblotted with anti-Flag antibodies to detect bound GRF4. As shown in FIG. 17, the full-length GRF4 binds specifically to GST-PDZ, showing that the interaction is mediated by binding of GST-PDZ to the SAV* motif present in the full-length GRF4. Furthermore, in a similar pull-down experiment, the streptavidin agarose beads bound to biotinylated peptide corresponding the last 15 amino acids of GRF4 (therefore, containing the SAV* motif) were shown to bind to the full-length GRF4 also, thus showing again an interaction between the PDZ domain and the SAV* motif of GRF4 (FIG. 18).

viii) Immunofluorescence studies/Localization

We determined that GRF4 exhibits plasma-membrane staining and is localized at the plasma membrane where Ras, its substrate, is located. This plasma membrane localization is mediated by the PDZ domain because the protein is localized diffusely in the cytosol upon mutation (eg. deletion of the PLPF domain) of the PDZ domain.

ix) Rap1 activation

We found that GRF4 mediates activation of Rap1 in cells via its CDC25 domain (FIG. 23f). Unlike Ras activation by GRF4, however, the activation of Rap1 was not stimulated by 8Br-cAMP (FIG. 23f), showing that GRF4mediated activation of Rap1 is constitutive and independent of cAMP stimulation. This is consistent with Ohtsuka et al. (T. Ohtsuka, et al., 1999).

Ras is localized at the plasma membrane and activation of SOS, GRF½ and GRP involves to some extent their translocation from the cytosol to the plasma membrane. Sos translocates to the plasma membrane following activation of tyrosine kinase (L. Buday, 1993), GRP in response to diacylglycerol production (J. O. Ebinu, et al., 1998, C. E. Tognon et al., 1998), and GRF2 in response to elevation of intracellular Ca²⁺ (N. Fam et al, 1997). Immunostaining of GRF4 transfected into HEK-293T cells revealed it is located at the periphery of the cell, showing it is targeted to the plasma membrane (FIG. 24a). This localization was not dependent on cAMP stimulation, showing that the cAMP-dependent activation of GRF4 (FIG. 23) was not related to translocation of the protein to the plasma membrane. Interestingly, this plasma membrane localization was impaired in GRF4 lacking an intact PDZ domain (missing the PLPF sequence, equivalent to the conserved GLGF sequence in many PDZ domains) (FIG. 24b), but not in cells expressing GRF4 bearing mutations in the PDZ-binding motif (SxV* changed to AAA*) (FIG. 24c). The PDZ domain is involved in targeting or tethering GRF4 to a PDZ-binding protein associated with the inner face of the plasma membrane.

x) Activation of Ras by GRF4:

We have already demonstrated that full-length GRF4 is active in catalyzing guanine-nucleotide exchange on small GTPase Ras using in-vitro GEF assay. As mentioned earlier, GRF4 has a REM domain which is present in all mammalian RasGRFs and therefore, we believe that GRF4 is a Ras-specific GRF. We test GRF4 activity on other small GTPases of Ras family (Ral, and so on) and those of Rho family (Rho, Rac and Cdc42) and show that GRF4 is a Ras and Rap1 specific GRF.

We also determine whether the GRF4-CDC25 domain is necessary and sufficient for its activity. First, we construct a mutant GRF4 construct lacking the CDC25 domain which can be expressed in mammalian cells and used in in-vitro GEF assays. This mutant construct, along with the full-length GRF4 which was already shown to be active on Ras, is measured for its activity or loss of activity. Furthermore, a GST-fusion protein of GRF4-CDC25 domain is generated and used in an in-vitro GEF assay to show that GRF4-CDC25 domain is sufficient for the GRF4 activity. GRF4 lacking the CDC25 domain will lose its ability to modulate Ras.

Concurrently, we measure the GEF activity on Ras of GRF4 on Ras in living cells, using the method described in Ref 35. This method employs a GST-fusion protein of Ras-binding domain (RBD) of Raf kinase (Raf is an immediate downstream kinase of Ras in MAPK pathway). Raf-RBD binds to Ras-GTP (active Ras) and thus is useful to assay levels of active Ras in cells. GST-RBD is incubated with lysates of cells transfected with GRF4 or empty vector. Active Ras in lystates is precipitated by GST-RBD beads and detected by anti-Ras antibodies on Western blot. In cells transfected with GRF4, we show more active Ras being pulled down by GST-RBD. This in vivo Ras activation assay also allows us to test effects of various treatments to cells of GRF4 activity.

We characterize the activation mechanisms of GRF4 and the signaling pathways employed by GRF4 from these in vivo Ras activation assays. For instance, since GRF4 has a cNMP-binding domain (cAMP-BD or cGMP-BD) we showed that cAMP or cAMP analogues activate GRF4. We construct a GST-fusion protein of this cAMP-BD in order to demonstrate its in-vitro binding affinity towards cAMP or cAMP using protocol previously described in Ref. 36.

We determine the roles of individual domains of GRF4 in Ras activation. We construct various mutant GRF4 constructs lacking individual domains which are tested for their activities on Ras using both in-vitro GEF assay and in vivo Ras activation assay.

The small GTPase Ras controls the MAPK pathway and exerts its effects on cellular processes primarily through this pathway. MAPK is a downstream kinase of Ras and thus, Ras activation leads to MAPK activation (FIG. 6A). Therefore, we show the GRF4 effects on MAPK activation using assays in which levels of active MAPK in cells is determined using antibodies recognizing phosphorylated (active) MAPK.

xi) Transforming ability of GRF4:

We already showed that GRF4 induces Rat 2 fibroblasts to form foci which are indicative of a loss of cell-cell contact inhibition. We use a mutant GRF4 construct lacking the catalytic domain which is therefore enzymatically inactive in the transformation assays alongside with the full-length GRF4 construct, in order to show that the CDC25 domain is necessary for the observed phenotype.

A loss of cell-ll contact inhibition and anchorage-independent growth are the two hallmarks of cellular transformation. These two properties underline the mechanism of tumor formation and metastasis. The oncogenic Ras and other oncogenes were already shown to exhibit these two transforming properties. We perform soft-agar assays to measure GRF4 anchorage-independent growth in Rat 2 cells transfected with GRF4.

We study the transforming ability of GRF4 in living animals. Tumor-formation assay is performed in nude mice ectopically injected with GRF4-induced transformed Rat2 cells.

xii) The activation mechanisms and signaling pathways employed by GRF4:

Although all known mammalian RasGRFs are activated by different signals arising from distinct signaling pathways (FIG. 6B), they all appear to employ similar activation mechanisms once they are recruited to the plasma membrane (where Ras is localized) in response to activating signals. Thus, membrane recruitment is a necessary step (however, it may not be sufficient) for activation of RasGRFs.

Localization studies of GRF4 are important in determining the activation mechanisms of this protein. We have performed immunofluorescence localization studies in Hek 293T cells transiently transfected with GRF4, using GRF4 specific antibodies which we have raised. Our results show that GRF4 is primarily associated with the plasma membrane. GRF4 has a PDZ domain and a PDZ-binding motif. PDZ domains have been known to be important in targeting proteins to the plasma membrane. Therefore, the PDZ domain of GRF4 targets K to the plasma membrane by likely binding to transmembrane receptors or ion channels which harbour its binding sites. The PDZ-binding motif of GRF4 does not mediate membrane targeting. We used mutant constructs either lacking the PDZ domain or having the PDZ-binding motif deleted in immunofluorescence localization studies to characterize their roles in GRF4 localization. We also perform localization studies on cells which are treated with various stimuli such as growth factors, cNMP-elevating agents, intracellular calcium elevating agents and so on, in order to measure each stimuli's effects on the localization of GRF4.

Our previous results from the binding studies with GRF4-PDZ domain show an intramolecular interaction in GRF4 by the association of its PDZ domain and its own PDZ-binding motif. If such an intramolecular interaction in GRF4 is used to regulate its activity, then the mutant constructs, which either lacks the PDZ domain or has the mutated PDZ-binding motif, affects GRF4 activity.

Since GRF4 has a cNMP-binding domain it shows that cNMP (preferably cAMP or cGMP) has regulatory roles on GRF4 activity and our recent work has shown activation of Ras/MAPK pathway by GRF4 in response to cAMP or cGMP analogues. We performed cNMP binding assays to show cAMP and cGMP binding to this domain. Cyclic AMP is a secondary messenger for G-protein coupled receptors which activate adenylyl cyclases by coupling to G-proteins. Many of these G-coupled receptors have PDZ-binding motifs in their intracellular C-terminal ends which potentially bind to PDZ-containing proteins. Having both a PDZ domain and a cAMP-binding domain, GRF4 may be involved in G-coupled receptor signaling pathways. We identify a receptor/receptors which bind specifically to the PDZ domain of GRF4 as binding leads to membrane targeting of GRF4 and to changes in GRF4 activity. We use several known G-coupled receptors such as beta-adrenergic receptors, Dopamine receptor and others. The later two are neuronal receptors and GRF4 was shown to be expressed strongly in the central nervous systems.

xiii) Determine the roles of Nedd4 in GRF4 regulation:

Since Nedd4 is a ubiquitin protein ligase, which we showed binds GRF4, it ubiquitinates and targets GRF4 for degradation. The mSOS2 was shown to be regulated by ubiquitination (46). We perform ubiquitination assays to measure GRF4 ubiquitination. Concurrently, stability studies (pulse-chase experiments) are also carried out to measure stability of GRF4.

In addition, since Nedd4 has a C2 domain which is a Ca2+-dependent lipid binding domain, we measure the effects of calcium on the localization and activity of GRF4.

MATERIALS AND METHODS

Identification of novel proteins interacting with Nedd4-WW domains

The method of identifying GRF4 is as follows. An expression library screen was used to identify proteins interacting with Nedd4-WW domains. GST-fusion proteins of individual WW domains of Nedd4 were constructed in pGEX2TK which contains a PKA phosphorylation site allowing radiolabeling of the fusion proteins with P32-ATP. The radiolabeled GST-fusion protein of Nedd4-WW2 domain was used as a probe to screen a 16-day mouse embryo expression library. About one million cDNA clones were screened. A total of 17 independent positive clones were isolated and sequenced using dideoxy sequencing method. All isolated clones contained at least one PY motif and thus are biochemically true positives.

Among the positive clones isolated was Clone 7.7. Clone 7.7 is a novel protein, the partial amino sequence of which exhibits 75% identity and 95% similarity of that of the novel human brain cDNA called KIAA0313. Because of this remarkable high sequence similarity between them, Clone 7.7 is the mouse homologue of KIAA0313 and obtained the full-length cDNA of KIAA0313.

Expression Cloning of GRF4

GST fusion protein encompassing the second WW domain of rat Nedd4 (GST-Nedd4-WW2), expressed in pGEX-2TK, was generated in bacteria. It was phosphorylated in vitro with bovine heart PKA (Sigma) and [³²Pγ]ATP, as described . A 16-day mouse embryo expression library (Novagen) was plated at a density of 3.5×10⁵ plaques per 150 mm plate and plaque-lifted onto isopropyl β-D-thiogalactoside-saturated nitrocellulose filters. Filters were then probed with the radiolabeled GST-Nedd4-WW2 fusion proteins.

GRF4 constructs and antibodies

Flag, HA or myc tags was added to the N-terminus of full length GRF4 using PCR, and subcloned into the mammalian expression vector pCMV5 (Invitrogen). Mutants GRF4 with deletion of the CDC25 domain (residue 711-1007, GRF4DCDC25 or DCDC25 for short), the cNMP-BD (residue 134-254, DcNMP-BD), the PLPF motif in the PDZ domain (residue 396-399, -PLPF), or bearing mutations in the PDZ-binding motif (SAV to AAA), were generated using PCR. The GST-GRF4-cNMP-BD (residue 101-303) and GST-Carboxy-terminus (residue 1348-1499) were PCR-generated and cloned into pGEX-2T ( Pharmacia). The latter construct was used to generate a fusion protein (GST-GRF4C terminus) which was used to immunize rabbits for the generation of polyclonal anti GRF4 antibodies.

Northern Blot Analysis

A 335 bp cDNA fragment corresponding to nucleotides 4286-4620 of KIAA0313 was labeled with [α³²P]dCTP by random priming using Random Primers DNA Labeling kit (Life Technologies). Both Rat Multiple Tissue and Human Brain Multiple Region blots (Clonetech) were probed in hybridization condition as previously described . The blots were washed for 30 min at 42° C. in 2×SSC/0.1% SDS and for 45 min at 55° C. in 0.1×SSC/0.1% SDS.

Cell Culture and Transfection

HEK-293T cells were maintained in Dulbecco's modified Eagle medium (DMEM) containing 10% fetal bovine serum, 100 Units of penicillin plus 100 μg of streptomycin per ml. Cells were transfected using the calcium phosphate precipitation method as described.

Cell Treatments

The membrane permeable 8-Br-cAMP and 8Br-cGMP analogs (Sigma) were used at a concenreation of 500 μM for 15 min. Inhibitors of PKA: H-89 and Rp-8-Br-cAMPS (CalBiochem) were used at 10 μM and 50 μM, respectively, for 30 min. Inhibitors of PKG: H-8 and Rp-8-Br-cGMPS (CalBiochem) were used at 5 μM and 25 μM, respectively, for 30 min. Activators of Adenylyl Cyclase, Forskolin (Sigma), and activator of Guanylyl Cyclase, YC-1 (CalBiochem), were used at 50 μM and 100 μM, respectively, for 15 min. Inhibitors of cAMP and cGMP phosphodiesterase (IBMX and Dipyrimidazole (CalBiochem)) were used at 100 μM and 10 μM, respectively, for 15 min.

Ras Activation Assay in living cells

HEK-293T cells were transfected as above, serum starved overnight and then subjected to various treatments, as described in the text. Cells were lysed with lysis buffer (25 mM Hepes, pH 7.5, 150 mM NaCl, 1% NP-40, 0.25% Na deoxycholate, 10% glycerol, 25 mM NaF, 10 mM MgCl₂, 1 mM EDTA, 1 mM NaVO₄, 10 mg/ml leupeptin, 10 mg/ml aprotinin, 250 mM PMSF) and the level of Ras.GTP or Rap. GTP in the lysates was determined using activation specific probes as described. Briefly, to determined the levels of active Ras (Ras-GTP) in cell, sepharose-bound GST fusion protein of the Ras-binding domain (RBD) of Raf-1 (GST-Raf1-RBD; Upstate Biotechnology Inc.) was used to precipitate Ras-GTP from cell lysates, and the amount of Ras-GTP determined by immunoblotting with anti Ras antibodies (Quality Biotech). Similarly, to determine the level of Rap1-GTP in cell lysates, sepharose-bound GST fusion proteins of the Rap1-binding domain of RaIGDS (GST-RaIGDS-RBD) were used to precipitate Rap1-GTP, which was then detected on a Western blot using anti-Rap1 antibodies (Transduction Lab).

Fluorescence Immunostaining

Transfected HEK-293T cells were fixed with 10% buffered Formalin phosphate (Fisher Scientific) for 30 min at 37° C., washed three times with PBS, permeabilized in TBS containing 1% Triton-X-100 for 10 min and blocked with blocking solution (TBS containing 5% goat serum (Gibco)) for 30 min. Fixed and permeabilized cells were then incubated with anti-GRF4 antibodies diluted in blocking solution for 1 h followed by four washes with TBS and incubation with FITC-conjugared goat anti-rabbit IgG. Stained cells were then visualized with a fluorescence microscope.

cAMP-agarose binding assay

cAMP-agarose (Sigma) was pre-incubated with PES containing 5 mg/ml BSA followed by incubation with either GST-GRF4-cNMP-BD or GST alone for in vitro binding assays, or with HEK-293T cell lysate expressing GRF4 or GRF4 lacking its cNMP binding domain (GRF4ΔcNMP-BD) for the pull down experiments. Following extensive washes in HNTG (20 mM Hepes, pH 7.5, 150 mM NaCl, 0.1% Triton ×100, 10% Glycerol) proteins were eluted off beads with sample buffer, separated on SDS-PAGE and immunoblotted with either anti GST antibodies for the in vitro binding experiments, or with anti GRF4 antibodies for the pull down experiments.

The present invention has been described in detail and with particular reference to the preferred embodiments: however, it will be understood by one having ordinary skill in the art that changes can be made without departing from the spirit and scope thereof. For example, where the application refers to proteins, it is clear that peptides and polypeptides may often be used. Likewise, where a gene is described in the application, it is clear that nucleic acid molecules or gene fragments may often be used.

All publications (including Genbank entries), patents and patent applications are incorporated by reference in their entirety to the same extent as if each individual publication, patent or patent application was specifically and individually indicated to be incorporated by reference in its entirety.

### REFERENCES

1- Staub, O., et al. The EMBO Journal 15, 2371-2380 ( 1996).

2- Gawler,D., Zhang, L.-J., Reedijk, M., Tung, P. S. & Moran, M. Oncogene 10, 817-825 (1995).

3- Perin, M. S., Brose, N., Jahn, R. & Sudof, T. C. Journal of Biological Chemistry 266, 623-629 (1991).

4- Shirataki, H., et al. Molecular and Cellular Biology 13, 2061-2068 (1993).

5- Clark, J. D., et al. Cell 65, 1043-1051 (1991).

6- Ponting, C. P. & Parker, P. J. Protein Science 5, 162-166 (1996).

7- Plant, P., et al. Journal of Biological Chemistry 272, 32329 (1997).

8- Andre, B. & Springael, J.-Y. Biochemical and Biophysical Reasearch Communications 205, 1201-1205 (1994).

9- Bork, P. & Sudol, M. Trends in Biochemical Sciences 19, (1994).

10- Hofmann, K. & Bucher, P. FEBS Letters 358, 153-157 (1995).

11- Staub, O. & Rotin, D. Structure 4, 495-499 (1996).

12- Macias, M. M., et al. Nature 382, 646-649 (1996).

13- Ranganathan, R., Lu, K., Hunter, T. & Noel, J. P. Cell 89,875-886 (1997).

14- Chen, H. I. & Sudol, M. Proceedings of the National Academy of Science (USA) 92, 7819-7823 (1995).

15- Shimkets, R. a., et al. Cell 79, 407-414 (1994).

16- Hansson, J. H., et al. P.N.A.S. (USA) 92.11495-11499 (1995).

17- Hansson, J. H., et al. Nature Genetics 11, 76-82 (1995).

18 Tamura, H., et al. Journal of Clinical investigation 97, 1780-1784 (1996).

19- Staub, O., et al. The EMBO Journal 16, 6325-6336 (1997).

20- Jung, D., et al. Journal of Biological Chemistry 270, 27305-27310 (1995).

21- Broek, D., et al. Cell 48, 789 (1987).

22- Camus, C., et al. Oncogene 11, 951-959 (1995).

23- Boriack-Sjodin, P.A., et al. Nature 394, 337-343 (1998).

24- Fam, N. P., et al. Molecular and Cellular Biology 17, 1396-1406 (1997).

25- Pawson, T. Nature 373, 573-580 (1995).

26- Farnsworth, C.L., et al. Nature 376, 524-527 (1995).

27- Ebinu J. O., et al. Science 280, 1082-1086 (1998).

28- Christopher P. Ponting, et al. BioEssays 19, 469-479 (1997).

29- Stephen N. Gomperts. Cell 84. 659-662 (1996).

30- Chevesich, J., et al. Neuron 18, 95-105 (1997).

31- Songyang, Z., et al. Science 275, 73-77 (1997).

32- Huang, L., et al. Nat Struct Biol 4(8), 609-615 (1997).

33- Phillip G. N. Protein 14, 425-9 (1992).

34- Trier, M., et at. Cell 78, 787-798 (1994).

35- Taylor, S. & Shalloway, D. Current Biology 6, 1621 ( 1996).

36- Doskeland, S. O., et al. Methods Enzymology 159, 147-150 (1988).

37- Hock, B., et al. PNAS 95, 9779-9784 (1998).

38- Wu, J., et al. Science 262, 1065-1068 (1993).

39- Cook, S. J., et al. Science 262, 1069-1071(1993).

40- Abriel, H., et al. (1998). J. Clin. Invest 103:667-673 (1999).

41- Bouras, M., et al. Eur. J. Endocrinol. 139, 209-16 (1998).

42- Matias, G., et al. Virchows Arch 433, 103-111 (1998).

43- Kressmen, U., et al. Eur. J. Cancer 34, 737-744 (1998).

44- Abrams, S. I., et al. Semin. Oncology 23, 118-134 (1996).

45- Chan, David C., et al. The EMBO Journal 15, 1045-1054 (1996).

46- Nielsen, K. H., et al. Mol. Cell. Biol. 17, 7132-8 (1997).

47-

48- Weisskopf, Marc G., et al. Science, Vol. 265,1878-1882 (1994).

49- Lyengar, Ravi. Science, Vol. 271, 461-463 (1996).

50- Shabb, John B., et al. The Journal of Biological Chemistry, Vol. 267, No. 9, 5723-5726 (199?).

51- Taylor, Susan S.. The Journal of Biological Chemistry, Vol. 264, No. 15, 8443-8446 (1989).

52- Frey, U., et al. Science, Vol. 260, 1661-1664 (1993).

53- Weber, Irene T., Biochemistry, 26, 343-351 (1987).

54- Hammerschmidt, Matthias, et al. Genes & Development, 10:647-658 (1996).

55- Bailey, Craig H., et al. Proc Natl. Acad. Sci. USA, Vol. 93, 13445-13452 (1996).

56- Downward, J., Nature, Vol. 396, 416-417 (1998).

57- Kawasaki, Kiroaki, et al. Science, Vol. 282, 2275-2279 (1998).

58- de Rool, Johan, et al. Nature, Vol. 396, 474-477 (1998).

59 Weber, et al. Biochemistry, 28:6122-6127. (1989).

60 Weber, et al. Biochemistry, 26:343-351(1987).

61 Su, etal. Science, 269:807-813 (1995).

62 O. Staub, et al., WW domains of Nedd4 bind to the proline-rich PY motifs in the epithelial Na+ channel deleted in Liddle's syndrome. Embo J 1996, 15, 2371-80.

63 H. Kawasaki, et al., A family of cAMP-binding proteins that directly activate Rap1. Science 1998, 282, 2275-9.

64 J. de Rooij, et al., Epac is a Rap1 guanine-nucleotide-exchange factor directly activated by cyclic AMP. Nature 1998, 396, 474-7.

65 P. A. Boriack-Sjodin, S. M. Margarit, D. Bar-Sagi, J. Kuriyan, The structural basis of the activation of Ras by Sos. Nature 1998, 394, 33743.

66 S. E. Craven, D. S. Bredt, PDZ proteins organize synaptic signaling pathways. Cell 1998, 93, 495-8.

67 Y. Su, et al., Regulatory subunit of protein kinase A: structure of deletion mutant with cAMP binding domains. Science 1995, 269, 807-13.

68 C. Herrmann, G. A. Martin, A. Wittinghofer, Quantitative analysis of the complex between p21ras and the Ras-binding domain of the human Raf-1 protein kinase. J Biol Chem 1995, 270, 2901-5.

69 J. de Rooij, J. L. Bos, Minimal Ras-binding domain of Raf1 can be used as an activation-specific probe for Ras. Oncogene 1997,14, 623-5.

70 S. J. Taylor, D. Shalloway, Cell cycle-dependent activation of Ras. Curr Biol 1996, 6, 1621-7.

71 T. Ohtsuka, et al., nRap GEP: A Novel Neural GDP/GTP Exchange Protein for Rap1 Small G. Protein That Interacts with Synaptic Scaffolding Molecule (S-SCAM). Biochem Biophys Res Commun 1999, 265, 38-44.

72 L. Buday, J. Downward, Epidermal growth factor regulates p21 ras through the formation of a complex of receptor, Grb2 adapter protein, and Sos nucleotide exchange factor. Cell 1993, 73, 611-620.

73 J. O. Ebinu, et al., RasGRP, a Ras guanyl nucleotide-releasing protein with calcium- and diacylglycerol-binding motifs. Science 1998, 280, 1082-6.

74 C. E. Tognon, et al., Regulation of RasGRP via a phorbol ester-responsive C1 domain. Mol Cell Biol 1998, 18, 6995-7008.

75 N. Fam, et al., Cloning and Characterization of Ras-GRF2, a novel guanine nucleotide exchange factor for Ras.. Mol Cell Biol, 1997, 17, 1396-1406. .

76 J. L. Bos, All in the family? New insights and questions regarding interconnectivity of Ras, Rap1 and Ral. Embo J 1998, 17, 6776-82.

References for Material and Methods

1. O. Staub, et al., WW domains of Nedd4 bind to the proline-rich PY motifs in the epithelial Na+ channel deleted in Liddle's syndrome. Embo J 1996, 15, 2371-80.

2. W. G. Kaelin, Jr., et al., Expression cloning of a cDNA encoding a retinoblastoma-binding protein with E2F-like properties. Cell 1992, 70, 351-64.

3. H. I. Chen, M. Sudol, The WW domain of Yes-associated protein binds a proline-rich ligand that differs from the consensus established for Src homology 3-binding modules. Proc Natl Acad Sci USA 1995, 92, 7819-23.

4. F. M. Ausubel, Current Protocols in Molecular Biology. 1987.

5. C. Chen, H. Okayama, High-efficiency transformation of mammalian cells by plasmid DNA. Mol Cell Biol 1987, 7, 2745-52.

6. N. Fam, et al., Cloning and Characterization of Ras-GRF2, a novel guanine nucleotide exchange factor for Ras., Mol Cell Biol, 1997, 17, 1396-1406..

7 S. J. Taylor, D. Shalloway, Cell cycle-dependent activation of Ras. Curr Biol 1996, 6, 1621-7.

8. J. de Rooij, J. L. Bos, Minimal Ras-binding domain of Raf1 can be used as an activation-specific probe for Ras. Oncogene 1997, 14, 623-5.

9. F. J. Zwartkruis, R. M. Wolthuis, N. M. Nabben, B. Franke, J. L. Bos, Extracellular signal-regulated activation of Rap1 fails to interfere in Ras effector signalling. Embo J 1998,17, 5905-12.

The inclusion of references in this application is not an admission that they are prior art.

We claim:

1. An isolated nucleic acid molecule encoding a polypeptide having GRF4 activity.

2. The nucleic acid molecule of claim 1, comprising all or part of the nucleic acid molecule of [SEQ ID NO: 1].

3. An isolated nucleic molecule comprising at least 40% sequence identity to all or part of the nucleic acid molecule of [SEQ ID NO: 1], wherein the nucleic acid molecule encodes a polypeptide having GRF4 activity.

4. The molecule of any of claims 1 to 3 which is selected from a group consisting of mRNA, cDNA, sense DNA, anti-sense DNA, single-stranded DNA and double-stranded DNA.

5. A nucleic acid molecule encoding the amino acid sequence of [SEQ ID NO: 2].

6. A nucleic acid molecule that encodes all or part of a GRF4 polypeptide or a polypeptide having GRF4 activity, wherein the sequence hybridizes to the nucleic acid molecule of all or part of [SEQ ID NO: 1] under high stringency conditions.

7. The nucleic acid molecule of claim 6, wherein the high stringency conditions comprise a wash stringency of about 0.2×SSC, about 0.1% SDS, at about 50-65° C.

8. An isolated polypeptide having GRF4 activity and a CDC25 domain.

9. The polypeptide of claim 8, comprising all or part of the sequence of [SEQ ID NO: 2].

10. An isolated polypeptide comprising at least 40% sequence identity to all or part of the polypeptide of [SEQ ID NO: 2], wherein the polypeptide has GRF4 activity.

11. A mimetic of the isolated polypeptide of any of claims 8 to 10, wherein the mimetic has GRF4 activity.

12. A recombinant nucleic acid molecule comprising a nucleic acid molecule of any of claim 1 to claim 7 and a promoter region, operatively linked so that the promoter enhances transcription of the nucleic acid molecule in a host cell.

13. A system for the expression of GRF4, comprising an expression vector and a nucleic acid molecule of any of claim 1 to claim 7 inserted in the expression vector.

14. The system of claim 13, wherein the expression vector comprises a plasmid or a virus.

15. A cell transformed by the expression vector of claim 14.

16. A method for expressing a polypeptide comprising: transforming an expression host with an expression vector including and culturing the expression host.

17. The method of claim 16, further comprising isolating the polypeptide.

18. The method of claim 16 or 17, wherein the expression host is selected from the group consisting of a plant, plant cell, bacterium, yeast, fungus, protozoa, algae, animal and animal cell.

19. A pharmaceutical composition, comprising all or part of the polypeptide or mimetic of any of claims 8 to 11, and a pharmaceutically acceptable carrier, auxiliary or excipient

20. A GRF4 specific antibody targeted to a region selected from the group consisting of the C-terminus, the CDC25 domain, the cNMP binding domain and the PDZ domain.

21. The antibody of claim 20, wherein the antibody is a monoclonal antibody or a polyclonal antibody.

22. A method of medical treatment of a disease, disorder or abnormal physical state, characterized by excessive GRF4 expression, concentration or activity, comprising administering a product that reduces or inhibits GRF4 polypeptide expression, concentration or activity.

23. The method of claim 22, wherein the product is an antisense nucleic acid molecule to all or part of the nucleic acid molecule of any of claims 1 to 7, the antisense nucleic acid molecule being sufficient to reduce or inhibit GRF4 polypeptide expression.

24. The method of claim 22, wherein the product comprises all or part of Nedd4.

25. The method of any of claims 22 to 24 wherein the disease, disorder or abnormal physical state comprises cancer.

26. A method of medical treatment of a disease, disorder or abnormal physical state, characterized by inadequate GRF4 expression, concentration or activity, comprising administering a product that increases GRF4 polypeptide expression, concentration or activity.

27. The method of claim 26, wherein the product is a nucleic acid molecule comprising all or part of the nucleic acid molecule of any of claims 1 to 7, the DNA being sufficient to increase GRF4 polypeptide expression.

28. The method of claim 27, wherein the nucleic acid molecule is administered in a pharmaceutical composition comprising a carrier and a vector operably linked to the nucleic acid molecule.

29. A method of identifying a compound which modulates the interaction of GRF4 with Ras, comprising
a) contacting (i) GRF4, a Ras-binding fragment of GRF4 or a derivative of either of the foregoing with (ii) Ras, a GRF4-binding fragment of Ras or a derivative of either of the foregoing in the presence of the compound; wherein (i) and (ii) are capable of binding; and
b) determining whether the binding between (i) and (ii) is modulated, thereby indicating that the compound modulates the interaction of GRF4 and Ras.

30. A method of identifying a compound which modulates the interaction of GRF4 with Rap1, comprising
a) contacting (i) GRF4, a Rap1-binding fragment of GRF4 or a derivative of either of the foregoing with (ii) Rap1, a GRF4-binding fragment of Rap1 or a derivative of either of the foregoing in the presence of the compound; wherein (i) and (ii) are capable of binding; and
b) determining whether the binding between (i) and (ii) is modulated, thereby indicating that the compound modulates the interaction of GRF4 and Rap1.

31. A method of evaluating the cell proliferation reducing properties of a compound comprising contacting the compound with:
a) GRF4, a Ras binding fragment of GRF4 or a derivative of either of the foregoing; and
b) Ras, a GRF4 binding fragment of Ras or a derivative of either of the foregoing; wherein (a) and (b) are capable of binding; and
c) determining the ability of the compound to interfere with the binding of a) with b), the ability to interfere with binding indicating that the compound reduces cell proliferation.

32. An isolated Guanine Nucleotide Releasing Factor 4 (GRF4) polypeptide Ras activator.

33. A recombinant GRF4 protein produced by a cell including a nucleic acid molecule encoding a GRF4, operably linked to a promoter.

34. A Ras binding peptide comprising 10 to 100 amino acids wherein the peptide includes part of the peptide of [SEQ. ID NO. 2, 4, 5 or 6] or a derivative thereof and inhibits Ras activation.

35. A method of evaluating an anti-proliferative compound comprising contacting the compound with the CDC25 domain of GRF4, or a derivative thereof and determining the ability of the compound to bind to the GRF4 or derivative, wherein the ability to bind indicates that the compound inhibits cell proliferation.


SEQUENCE LISTING (27 sequences)

SEQ ID NO: 1 - DNA, 6568 bp, Homo sapiens
  CDS (63)..(4562)
cttgccatcgtgagagattggtacatgatgtgtaaattcagttcagcatatgtttcttcattatgaaaccactagcaatcccagctaaccatggagttatgggccagcaggagaaacactcacttcctgcagatttcacaaaactgcatcttactgacagtctccacccacaggtgacccacgtttcttctagccattcaggatgtagtatcactagtgattctgggagcagcagtctttctgatatctaccaggccacagaaagcgaggctggtgatatggacctgagtgggttgccagaaacagcagtggattccgaagacgacgacgatgaagaagacattgagagagcatcagatcctctgatgagcagggacattgtgagagactgcctagagaaggacccaattgaccggacagatgatgacattgaacaactcttggaatttatgcaccagttgcctgcttttgccaatatgacaatgtcagtgaggcgagaactctgtgctgtgatggtgttcgcagtggtggaaagagcagggaccatagtgttaaatgatggtgaagagctggactcctggtcagtgattctcaatggatctgtggaagtgacttatccagatggaaaagcagaaatactgtgcatgggaaatagttttggtgtctctcctaccatggacaaagaatacatgaaaggagtgatgagaacaaaggtggatgactgccagtttgtctgcatagcccagcaagattactgccgtattctcaatcaagtagaaaagaacatgcaaaaagttgaagaggaaggagagattgttatggtgaaagaacaccgagaacttgatcgaactggaacaagaaagggacacattgtcatcaagggtacctcagaaaggttaacaatgcatttggtggaagagcattcagtagtagatccaacattcatagaagactttctgttgacctataggacttttctttctagcccaatggaagtgggcaaaaagttattggagtggtttaatgacccgagcctcagggataaggttacacgggtagtattattgtgggtaaataatcacttcaatgactttgaaggagatcctgcaatgactcgatttttagaagaatttgaaaacaatctggaaagagagaaaatgggtggacacctaaggctgttgaatatcgcgtgtgctgctaaagcaaaaagaagattgatgacgttaacaaaaccatcccgagaagctcctttgccttttatcttacttggaggctctgagaagggatttggaatctttgttgacagtgtagattcaggtagcaaagcaactgaagcaggcttgaaacggggggatcagatattagaagtaaatggccaaaactttgaaaacattcagctgtcaaaagctatggaaattcttagaaataacacacatttatctatcactgtgaaaaccaatttatttgtatttaaagaacttctaacaagattgtcagaagagaaaagaaatggtgccccccaccttcctaaaattggtgacattaaaaaggccagtcgctactccattccagatcttgctgtagatgtagaacaggtgataggacttgaaaaagtgaacaaaaaaagtaaagccaacactgtgggaggaaggaacaagctgaaaaagatactcgacaagactcggatcagtatcttgccacagaaaccatacaatgatattgggattggtcagtctcaagatgacagcatagtaggattaaggcagacaaagcacatcccaactgcattgcctgtcagtggaaccttatcatccagtaatcctgatttattgcagtcacatcatcgcattttagacttcagtgctactcctgacttgccagatcaagtgctaagggtttttaaggctgatcagcaaagccgctacatcatgatcagtaaggacactacagcaaaggaagtggtcattcaggctatcagggagtttgctgttactgccaccccggatcaatattcactatgtgaggtctctgtcacacctgagggagtaatcaaacaaagaagacttccagatcagctttccaaacttgcagacagaatacaactgagtggaaggtattatctgaaaaacaacatggaaacagaaactctttgttcagatgaagatgctcaggagttgttgagagagagtcaaatttccctccttcagctcagcactgtggaagttgcaacacagctctctatgcgaaattttgaactctttcgcaacattgaacctactgaatatatagatgatttatttaaactcagatcaaaaaccagctgtgccaacctgaagagatttgaagaagtcattaaccaggaaacattttgggtagcatctgaaattctcagagaaacaaaccagctgaagaggatgaagatcattaagcatttcatcaagatagcactgcactgtagggaatgcaagaattttaactcaatgtttgcaatcatcagtggcctaaacctggcaccagtggcaagactgcgaacgacctgggagaaacttcccaataaatacgaaaaactatttcaagatctccaagacctgtttgatccttccagaaacatggcaaaatatcgtaatgttctcaatagtcaaaatctacaacctcccataatccctctattcccagttatcaaaaaggatctcaccttccttcacgaaggaaatgactcaaaagtagacgggctggtcaattttgagaagctaaggatgattgcaaaagaaattcgtcacgttggccgaatggcttcagtgaacatggaccctgccctcatgttcaggactcggaagaagaaatggcggagtttggggtctctcagccagggtagtacaaatgcaacagtgctagatgttgctcagacaggtggtcataaaaagcgggtacgtcgtagttcctttctcaatgccaaaaagctttatgaagatgcccaaatggctcgaaaagtgaagcagtacctttccaatttggagctagaaatggacgaggagagtcttcagacattatctctgcagtgtgagccagcaaccaacacattgcctaagaatcctggtgacaaaaagcctgtcaaatccgagacctctccagtagctccaagggcagggtcacaacagaaagctcagtccctgccacagccccagcagcagccaccaccagcacataaaatcaaccagggactacaggttcccgccgtgtccctttatccttcacggaagaaagtgcccgtaaaggatctcccaccttttggcataaactctccacaagctttaaaaaaaattctttctttgtctgaagaaggaagtttggaacgtcacaagaaacaggctgaagatacaatatcaaatgcatcttcgcagctttcttctcctcctacttctccacagagttctccaaggaaaggctatactttggctcccagtggtactgtggataatttttcagattctggtcacagtgaaatttcttcacgatccagtattgttagcaattcgtcttttgactcagtgccagtctcactgcacgatgagaggcgccagaggcattctgtcagcatcgtggaaacaaacctagggatgggcaggatggagaggcggaccatgattgaacctgatcagtatagcttggggtcctatgcaccaatgtccgagggccgaggcttatatgctacagctacagtaatttcttctccaagcacagaggaactttcccaggatcagggggatcgcgcgtcacttgatgctgctgacagtggccgtgggagctggacgtcatgctcaagtggctcccatgataatatacagacgatccagcaccagagaagctgggagactcttccattcgggcatactcactttgattattcaggggatcctgcaggtttatgggcatcaagcagccatatggaccaaattatgttttctgatcatagcacaaagtataacaggcaaaatcaaagtagagagagccttgaacaagcccagtcccgagcaagctgggcgtcttccacaggttactggggagaagactcagaaggtgacacaggcacaataaagcggaggggtggaaaggatgtttccattgaagccgaaagcagtagcctaacgtctgtgactacggaagaaaccaagcctgtccccatgcctgcccacatagctgtggcatcaagtactacaaaggggctcattgcacgaaaggagggcaggtatcgagagcccccgcccacccctcccggctacattggaattcccattactgactttccagaagggcactcccatccagccaggaaaccgccggactacaacgtggcccttcagagatcgcggatggtcgcacgatcctccgacacagctgggccttcatccgtacagcagccacatgggcatcccaccagcagcaggcctgtgaacaaacctcagtggcataaaccgaacgagtctgacccgcgcctcgccccttatcagtcccaagggttttccaccgaggaggatgaagatgaacaagtttctgctgtttgaggcacagacttttctggaagcagagcgagccacctgaaaggagagcacaagaagacgtcctgagcattggagccttggaactcacattctgaggacggtggaccagtttgcctccttccctgccttaaaagcagcatggggcttcttctccccttcttcctttcccctttgcatgtgaaatactgtgaagaaattgccctggcacttttcagactttgttgcttgaaatgcacagtgcagcaatcttcgagctcccactgttgctgcctgccacatcacacagtatcattccaaattccaagatcatcacaacaagatgattcactctggctgcacttctcaatgcctggaaggattttttttaatcttccttttagatttcaatccagtcctagcacttgatctcattgggataatgagaaaagctagccattgaactacttggggcctttaacccaccaaggaagacaaagaaaaacaatgaaatcctttgagtacagtgcttgtccacttgtttacaatgtcctccttttaaaaaaaaaaatgagtttaaagattttgttcagagagtaaatatatatccatttaatgattacagtattattttaaaccttaagtagggttgccagcctggtttctgaaaaaccaaatatgccggacagggtgtggccacaccaagaagacgggaagacctggcttgtgaccctggcttcccatgtccttctggtctcacccgcgaagtgccctatcctggaagtatgaaatgttagccaattaataccaagacacctcatctgctccttccccagtggatggggttcttctgtaaaactgtttgcacatggccaggggagggaactaggacccttgtgtcctgtctgagccttatggaggcaggacggtgtcattggcggatgtgtcctgctccattgagatggatggcaaaccccatttttaagttatatttctttgatttttgttaatttagaggtgtaggttttgttttttgttttttgtttttttttaagagaaacatttataactggatagcattgcagtgaaagcagcttgggatgttggagctaatgccagctgtttatactgctctttcaagacagcctccctttattgaattggcattagggaataaacaagcctttaaacgtgataaaagatcaaaaacctggttagacatgccagcctttgcaaggcaggttagtcaccaaagactaacctccaagtggctttatggacgctgcatatagagaaggcctaagtgtagcaaccatctgctcacagctgctattaaccctataatgactgaaatgacccctccactctatttttgtgttgttttgcacagactccggaaaagtgaaggctgccaatctgagtagtactcaaatgtgaggaactgctggtcttggattttttttccattaaattcagctgatcatattgatcagtagataaacgtaaatagcttcaaattttaaaagtggaattgcagtgttttttcactgtatcaaacaatgtcagtgctttatttaataattctcttctgtatcatggcatttgtctacttgcttattacattgtcaattatgcatttgtaattttacatgtaatatgcattatttgccagttttattatataggctatggacctcatgtgcatatagaaagacagaaatctagctctaccacaagttgcacaaatgttatctaagcattaagtaattgtagaacataggactgctaatctcagttcgctctgtgatgtcaagtgcagaatgtacaattaactggtgatttcctcatacttttgatactacttgtacctgtatgtcttttagaaagacattggtggagtctgtatcccttttgtatttttaatacaataattgtacatattggttatatttttgttgaagatggtagaaatgtactatgtttatgcttctacatccagtttgtacaagctggaaaataaataaatataacat

SEQ ID NO: 2 - protein, 1499 aa, Homo sapiens
MKPLAIPANHGVMGQQEKHSLPADFTKLHLTDSLHPQVTHVSSSHSGCSITSDSGSSSLSDIYQATESEAGDMDLSGLPETAVDSEDDDDEEDIERASDPLMSRDIVRDCLEKDPIDRTDDDIEQLLEFMHQLPAFANMTMSVRRELCAVMVFAVVERAGTIVLNDGEELDSWSVILNGSVEVTYPDGKAEILCMGNSFGVSPTMDKEYMKGVMRTKVDDCQFVCIAQQDYCRILNQVEKNMQKVEEEGEIVMVKEHRELDRTGTRKGHIVIKGTSERLTMHLVEEHSVVDPTFIEDFLLTYRTFLSSPMEVGKKLLEWFNDPSLRDKVTRVVLLWVNNHFNDFEGDPAMTRFLEEFENNLEREKMGGHLRLLNIACAAKAKRRLMTLTKPSREAPLPFILLGGSEKGFGIFVDSVDSGSKATEAGLKRGDQILEVNGQNFENIQLSKAMEILRNNTHLSITVKTNLFVFKELLTRLSEEKRNGAPHLPKIGDIKKASRYSIPDLAVDVEQVIGLEKVNKKSKANTVGGRNKLKKILDKTRISILPQKPYNDIGIGQSQDDSIVGLRQTKHIPTALPVSGTLSSSNPDLLQSHHRILDFSATPDLPDQVLRVFKADQQSRYIMISKDTTAKEVVIQAIREFAVTATPDQYSLCEVSVTPEGVIKQRRLPDQLSKLADRIQLSGRYYLKNNMETETLCSDEDAQELLRESQISLLQLSTVEVATQLSMRNFELFRNIEPTEYIDDLFKLRSKTSCANLKRFEEVINQETFWVASEILRETNQLKRMKIIKHFIKIALHCRECKNFNSMFAIISGLNLAPVARLRTTWEKLPNKYEKLFQDLQDLFDPSRNMAKYRNVLNSQNLQPPIIPLFPVIKKDLTFLHEGNDSKVDGLVNFEKLRMIAKEIRHVGRMASVNMDPALMFRTRKKKWRSLGSLSQGSTNATVLDVAQTGGHKKRVRRSSFLNAKKLYEDAQMARKVKQYLSNLELEMDEESLQTLSLQCEPATNTLPKNPGDKKPVKSETSPVAPRAGSQQKAQSLPQPQQQPPPAHKINQGLQVPAVSLYPSRKKVPVKDLPPFGINSPQALKKILSLSEEGSLERHKKQAEDTISNASSQLSSPPTSPQSSPRKGYTLAPSGTVDNFSDSGHSEISSRSSIVSNSSFDSVPVSLHDERRQRHSVSIVETNLGMGRMERRTMIEPDQYSLGSYAPMSEGRGLYATATVISSPSTEELSQDQGDRASLDAADSGRGSWTSCSSGSHDNIQTIQHQRSWETLPFGHTHFDYSGDPAGLWASSSHMDQIMFSDHSTKYNRQNQSRESLEQAQSRASWASSTGYWGEDSEGDTGTIKRRGGKDVSIEAESSSLTSVTTEETKPVPMPAHIAVASSTTKGLIARKEGRYREPPPTPPGYIGIPITDFPEGHSHPARKPPDYNVALQRSRMVARSSDTAGPSSVQQPHGHPTSSRPVNKPQWHKPNESDPRLAPYQSQGFSTEEDEDEQVSAV

SEQ ID NO: 3 - DNA, 799 bp, Mus musculus
actaaagggaacaaaagctggagctccaccgcggtggcggccgctctagaactagtggatcccccgggctgcaggaattcaagcggtgggaaggatgtctccgctgaggcagagagcagcagcatggtgcccgtgactacagaggaagccaaacctgtccctatgcctgcccacatagctgtgacgccgagcactaccaagggactcatcgcacggaaggaaggcaggtaccgggagccgcctcccacacctccaggctacgtgggcatccccattgccgatttcccagaagggccttgccacccggccaggaagcccccggattacaacgtggccctgcagcggtcccgcatggtggcacggcccactgaggccccggcaccgggccagacgccgcctgcagccgcagccagccggccgggcagcaagccacagtggcacaagcccagcgacgcagacccacgcctcgcgcccttccaggcaggcttcgcaggagcggaggaggacgaagatgaacaagtgtctgctgtttgaggcgcaggctccttgatccacagtgagccacccaaaggagagcacaagaagacgtcccaagccttggagccttggcacgcacatctgaggatggtggaccagtttgcctccttccctgccttaaagcagcatggggcttcttctccccttcttcctttcccctttgcatgtgaaatactgtgaagaaattgccctggcactttgcagacttgttgcttgaaatgcacagcccagcagcccctgagctgctgcctgccacgtcacg

SEQ ID NO: 4 - protein, 286 aa, Homo sapiens
  SIMILAR: Xaa is any aa
TKGNKSWSSTAVAAALELVDPPGCRNSSGGKDVSAEAESSSMVPVTTEEAKPVPMPAHIAVTPSTTKGLIARKEGRYREPPPTPPGYVGIPIADFPEGPCHPARKPPDYNVALQRSRMVARPTEAPAPGQTPPAAAASRPGSKPQWHKPSDADPRLAPFQAASHSGTSPATQTHASRPSRQASQERRRTKMNKCLLFEAQAPXSTVSHPKESTRRRPKPWSLGTHIXGWWTSLPPSLPXSSMGLLLPFFLSPLHVKYCEEIALALCRLVAXNAQPSSPXAAACHVT

SEQ ID NO: 5 - protein, 245 aa, Homo sapiens
  SIMILAR: Xaa is any aa
LKGTKAGAPPRWRPLXNXWIPRAAGIQAVGRMSPLRQRAAAWCPXLQRKPNLSLCLPTXLXRRALPRDSSHGRKAGTGSRLPHLQATWASPLPISQKGLATRPGSPRITTWPCSGPAWWHGPLRPRHRARRRLQPQPAGRRLRRSGGGRRXTSVCCLRRRLLDPQXATQRRAQEDVPSLGALARTSEDGGPVCLLPCLKAAWGFFSPSSFPLCMXNTVKKLPWHFADLLLEMHSPAAPELLPATS

SEQ ID NO: 6 - protein, 266 aa, Homo sapiens
  SIMILAR: Xaa is any aa
XREQKLELHRGGGRSRTSGSPGLQEFKRWEGCLRXGREQQHGARDYRGSQTCPYACPHSCDAEHYQGTHRTEGRQVPGAASHTSRLRGHPHCRFPRRALPPGQEAPGLQRGPAAVPHGGTAHXGPGTGPDAACSRSQPAGQQATVAQAQRRRPTPRALPGAGFAGAEEDEDEQVSAVXGAGSLIHSEPPKGEHKKTSQALEPWHAHLRMVDQFASFPALKQHGASSPLLPFPFACEILXRNCPGTLQTCCLKCTAQQPLSCCLPRH

SEQ ID NO: 7 - protein, 307 aa, Drosophila melanogaster
SNVHFLHLNAYELAIQLTLQDFANFRQIESTEYVDELFELRSRYGVPMLSKFAELVNREMFWVVSEICAEHNIVRRMKIVKQFIKIARHCKECRNFNSMFAIVSGLGHGAVSRLRQTWEKLPSKYQRLFNDLQDLMDPSRNMSKYRQLVSAELLAQHPIIPFYPIVKKDLTFIHLGNDTRVDGLVNFEKLRMLAKEVRLLTHMCSSPYDLLSILELKGQSPSNALFSLNQMSASQSNAAAGTVIAANAGQATIKRRKKSTAAPNPKKMFEEAQMVRRVKAYLNSLKILSDEDLLHKFSLECEPAHGS

SEQ ID NO: 8 - protein, 270 aa, Homo sapiens
SAEGLDLVSAKDLAGQLTDHDWSLFNSIHQVELIHYVLGPQHLRDVTTANLERFMRRFNELQYWVATELCLCPVPGPRAQLLRKFIKLAAHLKEQKNLNSFFAVMFGLSNSAISRLAHTWERLPHKVRKLYSALERLLDPSWNHRVYRLALAKLSPPVIPFMPLLLKDMTFIHEGNHTLVENLINFEKMRMMARAARMLHHCRSHNPVPLSPLRSRVSHLHEDSQVARISTCSEQSLSTRSPASTWAYVQQLKVIDNQRELSRLSRELEP

SEQ ID NO: 9 - protein, 244 aa, Mus musculus
KAECFETLSAMELAEQITLLDHIVFRSIPYEEFLGQGWMKLDKNERTPYIMKTSQHFNEMSNLVASQIMNYADISSRPNAIEKWVAVADICRCLHNYNGVLEITSALNRSPIYRLKKTWAKVSKQTKALMDKLQKTVSSEGRFKNLRETLKNCNPPAVPYLGMYLTDLAFIEEGTPNFTEEGLVNFSKMRMISHIIREIRQFQQTAYRIDQQPKVIQYLLDKALVIDEDSLYELSLKIEPRLPA

SEQ ID NO: 10 - protein, 249 aa, Homo sapiens
DEITLLTLHPLELARQLTLLEFEMYKNVKPSELVGSPWTKKDKEVKSPNLLKIMKHTTNVTRWIEKSITEAENYEERLAIMQRAIEVMMVMLELNNFNGILSIVAAMGTASVYRLRWTFQGLPERYRKFLEECRELSDDHLKKYQERLRSINPPCVPFFGRYLTNILHLEEGNPDLLANTELINFSKRRKVAEIIGEIQQYQNQPYCLNEESTIRQFFEQLDPFNGLSDKQMSDYLYNESLRIEPRGCK

SEQ ID NO: 11 - protein, 243 aa, Homo sapiens
VSLLFDHLEPEELSEHLTYLEFKSFRRISFSDYQNYLVNSCVKENPTMERSIALCNGISQWVQLMVLSRPTPQLRAEVFIKFIQVAQKLHQLQNFNTLMAVIGGLCHSSISRLKETSSHVPHEINKVLGEMTELLSSSRNYDNYRRAYGECTDFKIPILGVHLKDLISLYEAMPDYLEDGKVNVHKLLALYNHISELVQLQEVAPPLEANKDLVHLLTLSLDLYYTEDEIYELSYAREPRNHR

SEQ ID NO: 12 - protein, 48 aa, Unknown Organism
  Description of Unknown Organism  unavailable
IRGGTKEALIEHLTSHELVDAAFNVTMLITFRSILTTREFFYALIYRY

SEQ ID NO: 13 - protein, 47 aa, Mus musculus
IKGGTVVKLIERLTYHMYADPNFVRTFLTYRSFCKQELLNLLIERFE

SEQ ID NO: 14 - protein, 48 aa, Mus musculus
IRYASVEALLERLTDLRFLSIDFLNTFLHTYRIFTTATVVLAKLSDIY

SEQ ID NO: 15 - protein, 50 aa, Unknown Organism
  SIMILAR: Xaa is any aa
VVKFASLNKLVEHLTHDSKHDLQFLKTFLMTYQSFCTPEKLMSKLQQRYX

SEQ ID NO: 16 - protein, 77 aa, Drosophila melanogaster
LTRSSRDEPLNFRIVGGYELRGVAIATGNAAVGIYISHVEPGSKAQDVGLKRGDQIHEVNGQSLDHVTSKRALEILT

SEQ ID NO: 17 - protein, 71 aa, Homo sapiens
NLKKDAKYGLGFQIIGGEKMGRLDLGIFISSVAPGGPADLDGCLKPGDRLISVNSVSLEGVSHHAAIEILQ

SEQ ID NO: 18 - protein, 67 aa, Homo sapiens
IVIHRGSTGLGFNIVGGEDGEGIFISFILAGGPADLSGELRKGDQILSVNGVDLRNASHEQAAIALK

SEQ ID NO: 19 - protein, 68 aa, Rattus rattus
VELPKTEEGLGFNIMGGKEQNSPIYISRIIPGGIADRHGGLKRGDQLLSVNGVSVEGEHHEKAVELLK

SEQ ID NO: 20 - protein, 65 aa, Homo sapiens
VKVQKGSEPLGISIVSGEKGGIYVSKVTVGSIAHQAGLEYGDQLLEFNGINLRSATEQQARLIIG

SEQ ID NO: 21 - protein, 98 aa, Drosophila melanogaster
MVFAVVDKAGTVVMSDGEELDSWSVLINGAVEIEHANGSREELQMGDSFGILPTMDKLYHRGVMRTKCDDCQFVCITQTDYYRIQHQGEENTRRHEDE

SEQ ID NO: 22 - protein, 99 aa, Homo sapiens
LLFEPHSKAGTVLFSQGDKGTSWYIIWKGSVNVVTHGKGLVTTLHEGDDFGQLALVNDAPRAATIILREDNCHFLRVDKQDFNRIIKDVEAKTMRLEEH

SEQ ID NO: 23 - protein, 97 aa, Homo sapiens
AMFPVTHIAGETVIQQGNEGDNFYVVDQGEVDVYVNGEWVTNISEGGSFGELALIYGTPRAATVKAKTDLKLWGIDRDSYRRILMGSTLRKRKMYEE

SEQ ID NO: 24 - protein, 97 aa, Homo sapiens
CMYGRNYQQGSYIIKQGEPGNHIFVLAEGRLEVFQGEKLLSSIPMWTTFGELAILYNCTRTASVKAITNVKTWALDREVFQNIMRRTAQARDEQYRN

SEQ ID NO: 25 - protein, 103 aa, Mus musculus
RLRSVVYLPNDYVCKKGEIGREMYIIQAGQVQVLGGPDGKSVLVTLKAGSVFGEISLLAVGGGNRRTANVVAHGFTNLFILDKKDLNEILVHYPESQKLLRKK

SEQ ID NO: 26 - protein, 91 aa, Unknown Organism
  Description of Unknown Organism  unavailable
REDFEIIRVFDGNNSYRSQISRNIVVAKHVSVQQVRDAALRRFHINDTPERYYITQVVGEVEEEILEDPVPLRNVKRPEGKRAQIFIRYYD

SEQ ID NO: 27 - protein, 129 aa, Unknown Organism
  Description of Unknown Organism  unavailable
SILVTSQDKAPSVISRVLKKNNRDSAVASEYELVQLLPGERELTIPASANVFYAMDGASHDFLLRHGEGPLLLHLASPVARLPQELLRVREEGAPFPGSRPQGGRLHGHCSEEEAPLAYRSHGVHTRCG